(19) United States
(12) Patent Application Publication
(10) Pub. No.: US 2024/0287038 A1
(43) Pub. Date: Aug. 29, 2024

(54) QUINOXALINE COMPOUND AND APPLICATION THEREOF

(71) Applicant: Shanghai Tianma Microelectronics Co., Ltd., Shanghai (CN)

(72) Inventors: Kebin AN, Shanghai (CN); Wei GAO, Shanghai (CN); Wenpeng DAI, Shanghai (CN); Tingting LU, Shanghai (CN); Lu ZHAI, Shanghai (CN)

(73) Assignee: Shanghai Tianma Microelectronics Co., Ltd., Shanghai (CN)

(21) Appl. No.: 18/645,869

(22) Filed: Apr. 25, 2024

(30) Foreign Application Priority Data
Sep. 20, 2023 (CN) 202311224229.9

(51) Int. Cl. C07D 401/14; C07D 403/04; H10K 50/16; H10K 85/60; H10K 101/30

(52) U.S. Cl. CPC C07D 401/14; C07D 403/04; H10K 50/16; H10K 85/654; H10K 85/6572; H10K 2101/30

(57) ABSTRACT

A quinoxaline compound and application thereof are described. In an example, the quinoxaline compound has a high glass transition temperature and molecular thermal stability, suitable HOMO and LUMO energy levels, and high electron mobility, and can effectively improve luminous efficiency and service life of an organic electroluminescent device after being applied to preparation of the device.

### CROSS-REFERENCE TO RELATED APPLICATIONS

The present application claims priority to Chinese patent application no. 202311224229.9, filed on Sep. 20, 2023, the content of which is incorporated herein by reference in its entirety.

### TECHNICAL FIELD

The present disclosure belongs to the technical field of organic electroluminescence, and particularly relates to a quinoxaline compound and application thereof.

### BACKGROUND

Organic electroluminescence technology can be applied in the fields of display and lighting, is expected to replace existing liquid crystal display and fluorescent lamp illumination, and has a wide application prospect. The organic electroluminescent device has a sandwich liked structure, including electrode materials at both ends and organic functional materials sandwiched between the electrode materials. These materials are stacked together to form the organic electroluminescent device. When a voltage is applied to the electrodes at both ends of the organic electroluminescent device, carriers are injected into the organic functional material and transmitted through the action of an electric field, and finally the carriers recombine in the light-emitting layer, thereby generating electroluminescence.

Research on improving performance of organic electroluminescent devices mainly includes reducing driving voltage of the device, improving luminous efficiency of the device, and prolonging service life of the device. In order to achieve performance improvement, not only the device structure needs to be optimized and the device preparation process needs to be improved, developing high-performance organic functional material is more important. Improvement of the photoelectric performance of the electron transmission material, as one of organic functional materials, is critical for improving overall performance of the device. However, the conventional electron transmission material has low electron mobility, causing unbalanced transmission of electron and hole inside the device, thereby resulting in low device performance. On the other hand, the conventional electron transmission material has low glass transition temperature and poor thermal stability, and is prone to crystallization and degradation during extended operation of the device, resulting in performance degradation. For the actual requirements of the current display lighting industry, there is an important practical application value for designing and developing a stable and efficient electron transmission material and/or an electron injection material which can be effectively doped with metal Yb or LiQ, to reduce driving voltage, improve luminous efficiency, and prolong service life of the device.

### SUMMARY

The present disclosure provides a quinoxaline compound and application thereof. In an embodiment, the compound of the present disclosure contains quinoxaline and pyridine structures, and has high glass transition temperature and molecular thermal stability, suitable HOMO and LUMO energy levels, and high electron mobility, and which effectively improve luminous efficiency and service life of an organic electroluminescent device after being applied to preparation of the device.

Embodiments employ the following technical solution.

In an aspect, the present disclosure provides a quinoxaline compound having a structure represented by Formula I:

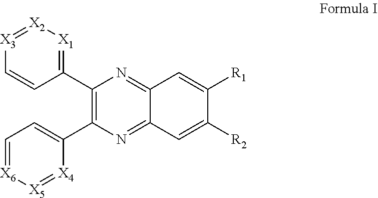

where R₁ and R₂ are each independently selected from hydrogen, substituted or unsubstituted C3-C60 aryl, and substituted or unsubstituted C3-C60 heteroaryl; X₁, X₂, X₃, X₄, X₅, and X₆ are each independently selected from methylene or nitrogen atom, and at least one of X₁, X₂, X₃, X₄, X₅, and X₆ contains an N atom.

In some embodiments, R₁ and R₂ are each independently selected from C3-C60 aza-aromatic group.

In some embodiments, R₁ and R₂ are the same or different.

In some embodiments, X₁, X₂, X₃, X₄, X₅, and X₆ are each independently selected from methylene or nitrogen atom, and one of X₁, X₂, X₃, X₄, X₅, and X₆ contains an N atom.

In some embodiments, R₁ and R₂ are each independently selected from any one of the following groups:

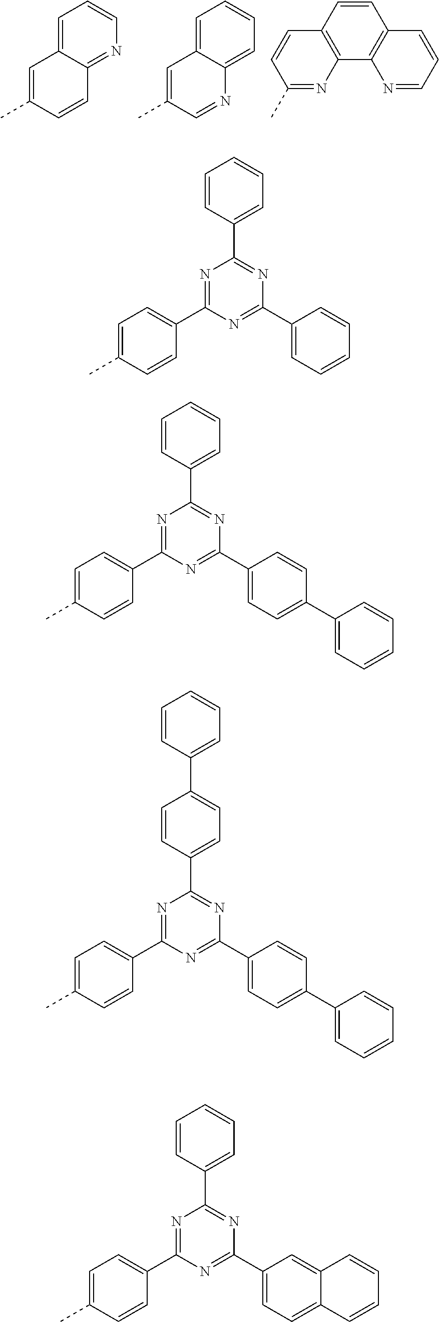

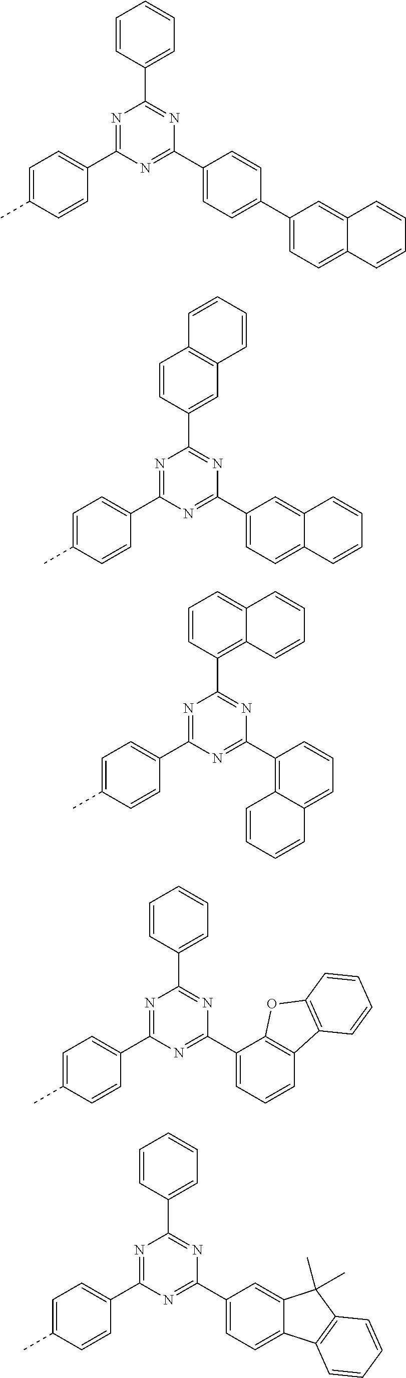

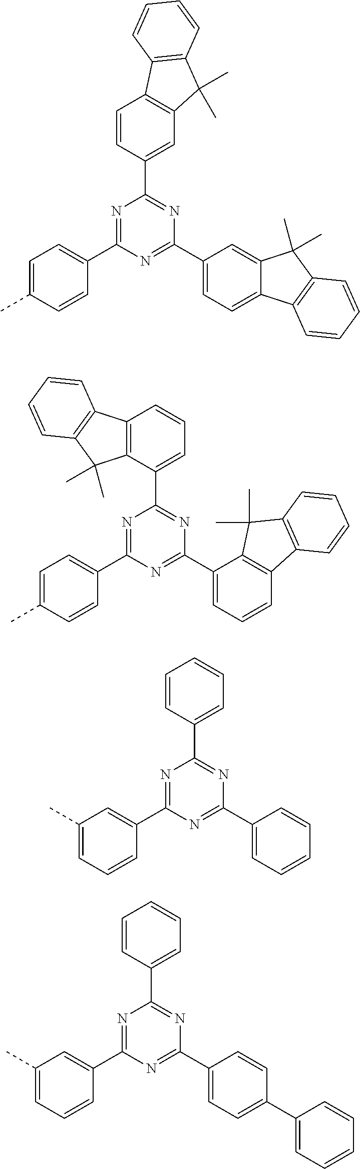

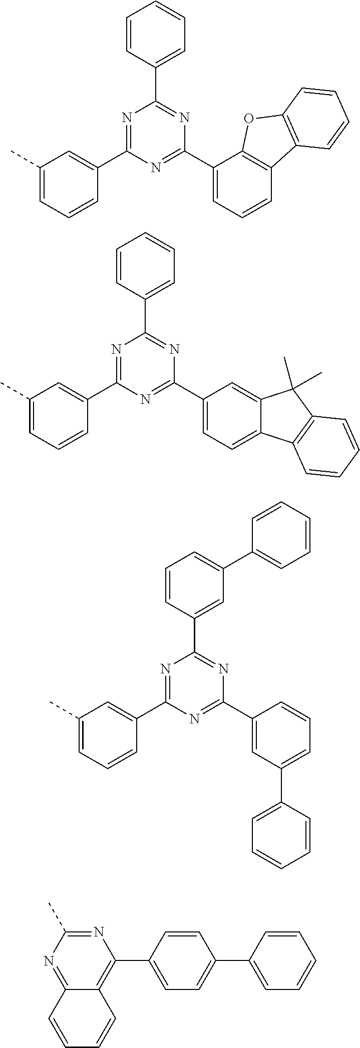

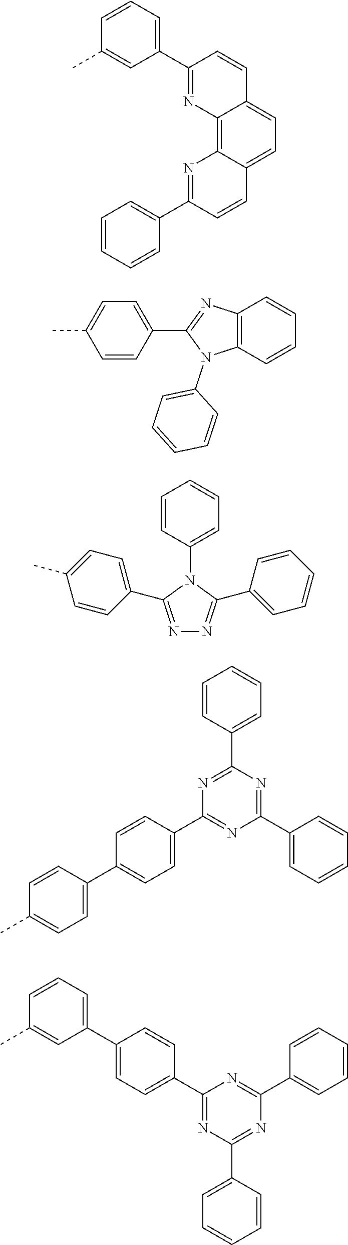

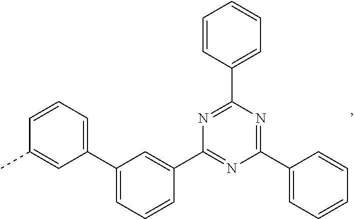

where the dashed line represents a bonding site of the group.

In some embodiments, the quinoxaline organic electroluminescent compound of the present disclosure is selected from the following chemical structures:

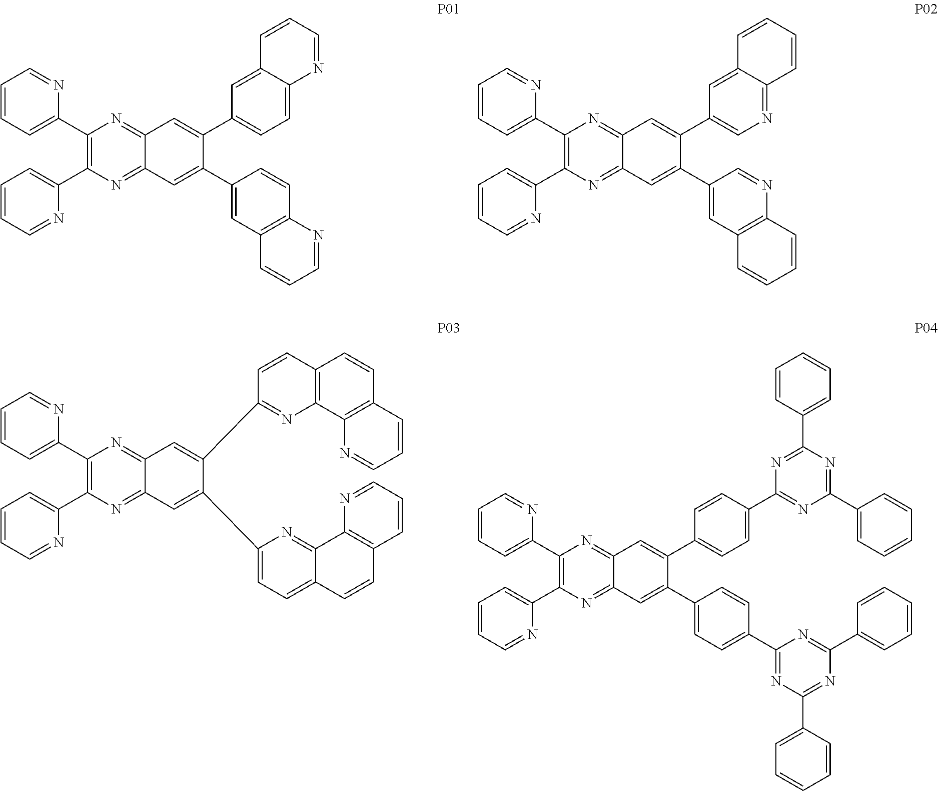 [SMILES: c1ccc(-c2nc3cc(-c4ccc5ncccc5c4)c(-c4ccc5ncccc5c4)cc3nc2-c2ccccn2)nc1 ; c1ccc(-c2nc3cc(-c4cnc5ccccc5c4)c(-c4cnc5ccccc5c4)cc3nc2-c2ccccn2)nc1 ; c1ccc(-c2nc3cc(-c4ccc5ccc6cccnc6c5n4)c(-c4ccc5ccc6cccnc6c5n4)cc3nc2-c2ccccn2)nc1 ; c1ccc(-c2nc(-c3ccccc3)nc(-c3ccc(-c4cc5nc(-c6ccccn6)c(-c6ccccn6)nc5cc4-c4ccc(-c5nc(-c6ccccc6)nc(-c6ccccc6)n5)cc4)cc3)n2)cc1]

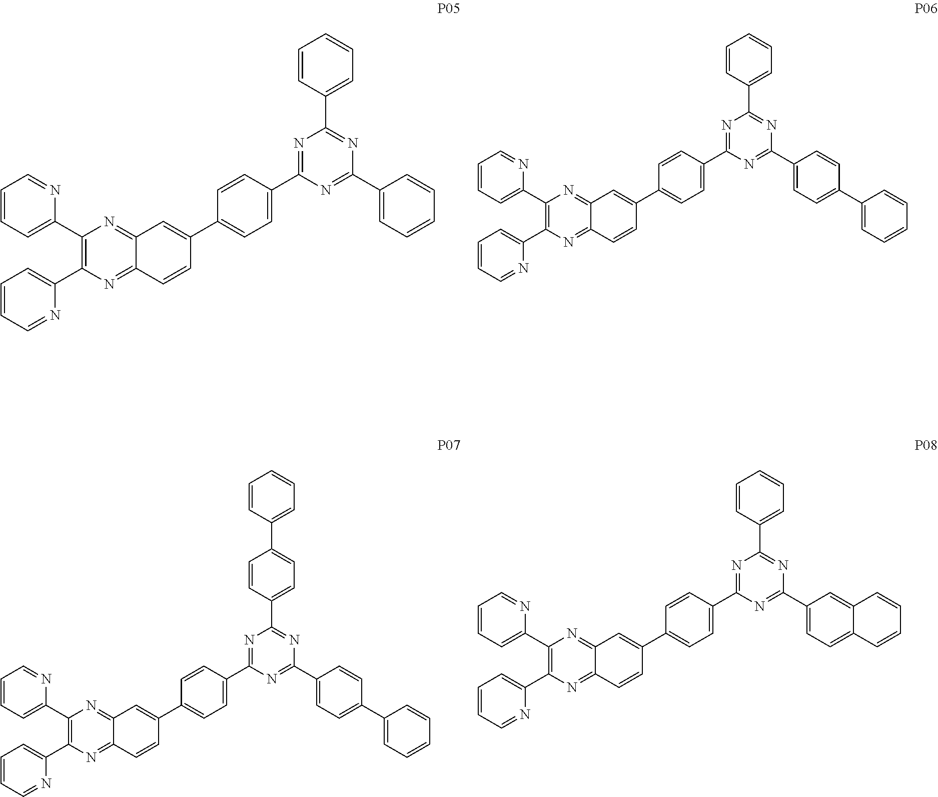 [SMILES: c1ccc(-c2nc(-c3ccccc3)nc(-c3ccc(-c4ccc5nc(-c6ccccn6)c(-c6ccccn6)nc5c4)cc3)n2)cc1 ; c1ccc(-c2ccc(-c3nc(-c4ccccc4)nc(-c4ccc(-c5ccc6nc(-c7ccccn7)c(-c7ccccn7)nc6c5)cc4)n3)cc2)cc1 ; c1ccc(-c2ccc(-c3nc(-c4ccc(-c5ccccc5)cc4)nc(-c4ccc(-c5ccc6nc(-c7ccccn7)c(-c7ccccn7)nc6c5)cc4)n3)cc2)cc1 ; c1ccc(-c2nc(-c3ccc(-c4ccc5nc(-c6ccccn6)c(-c6ccccn6)nc5c4)cc3)nc(-c3ccc4ccccc4c3)n2)cc1]

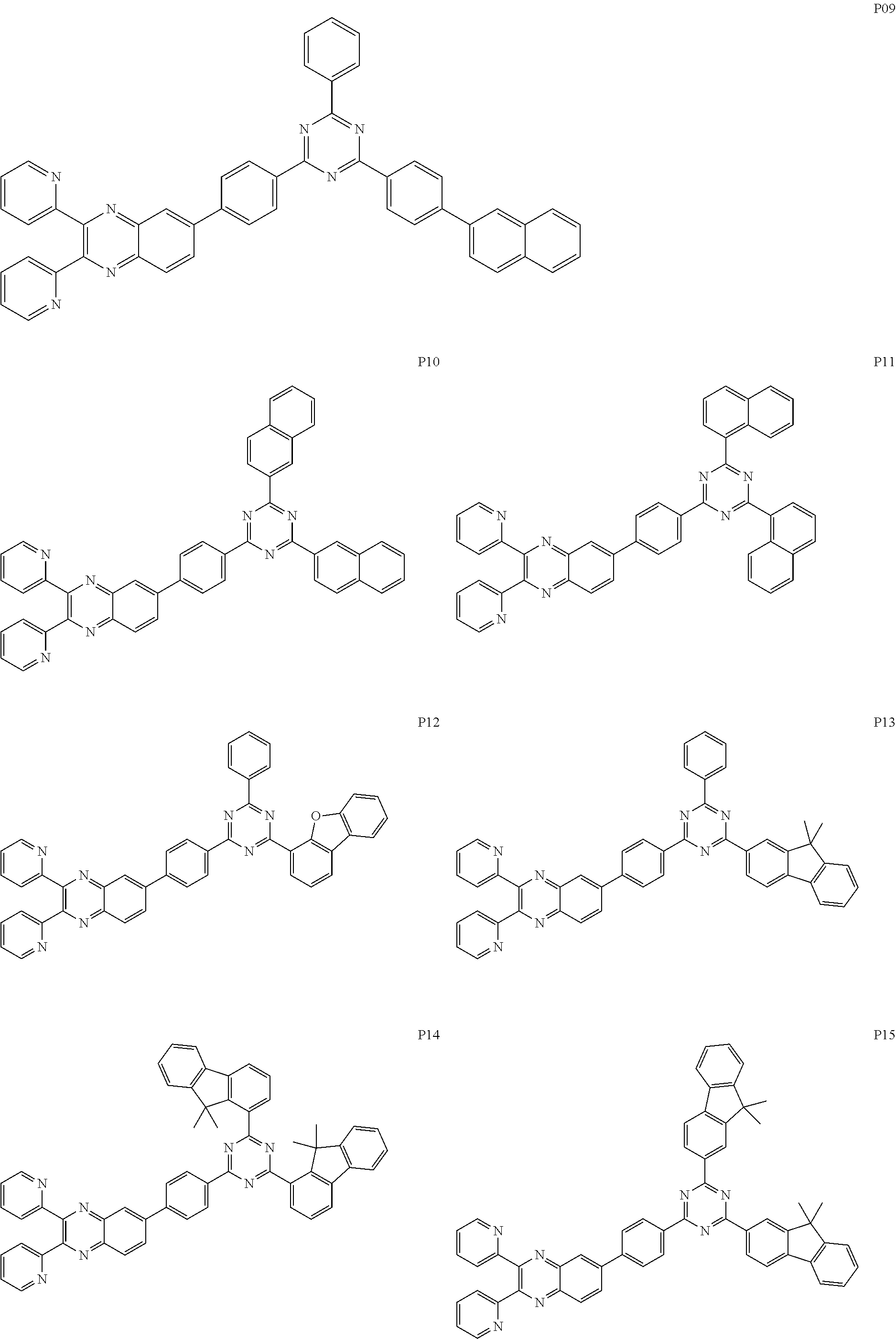

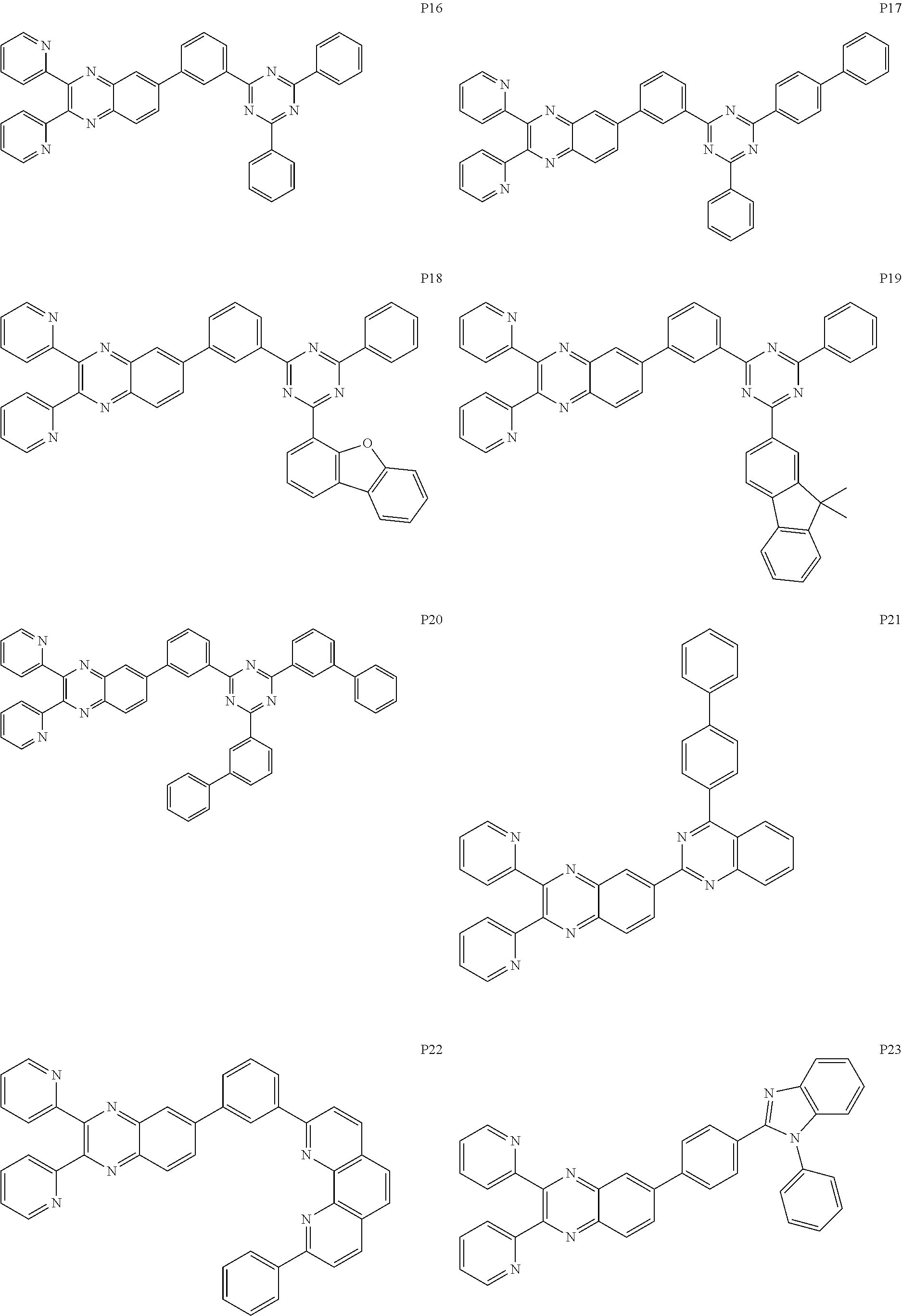

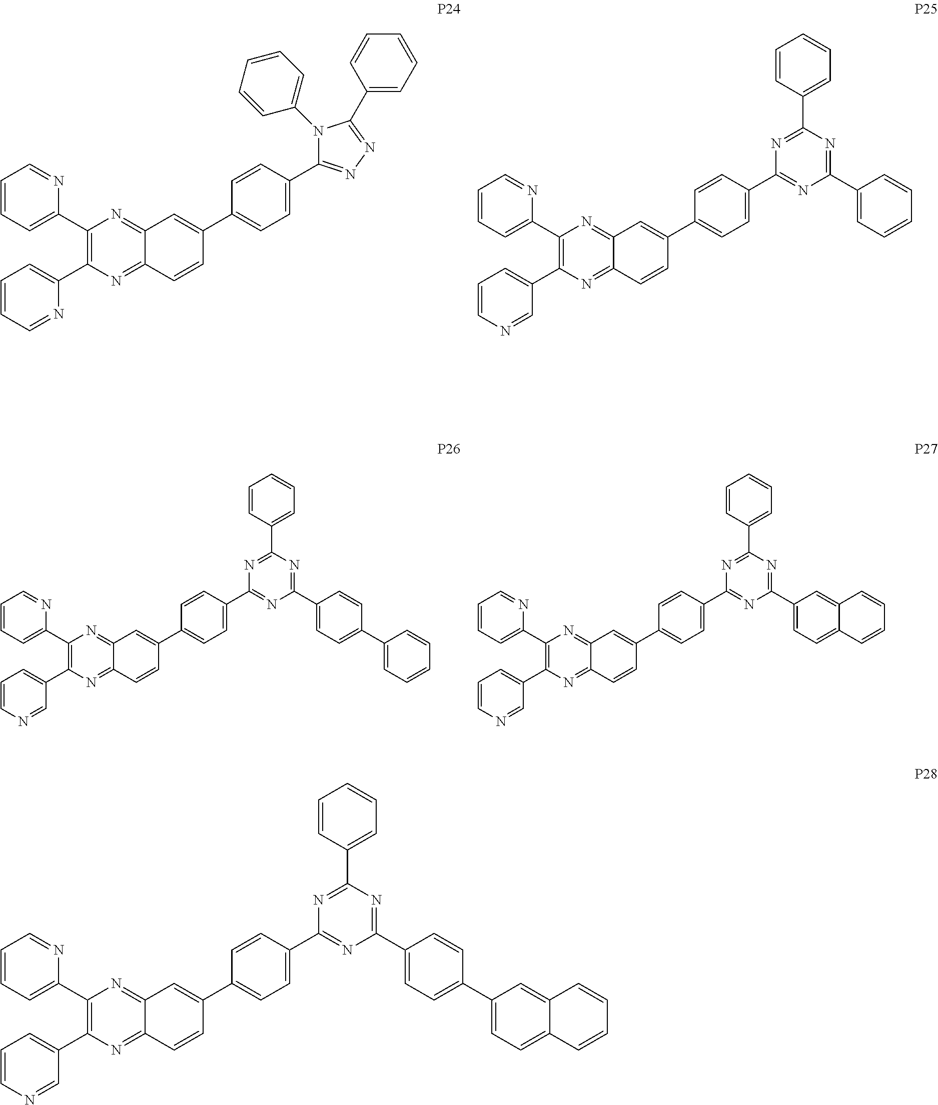 [SMILES: c1ccc(-c2nnc(-c3ccc(-c4ccc5nc(-c6ccccn6)c(-c6ccccn6)nc5c4)cc3)n2-c2ccccc2)cc1 ; c1ccc(-c2nc(-c3ccccc3)nc(-c3ccc(-c4ccc5nc(-c6cccnc6)c(-c6ccccn6)nc5c4)cc3)n2)cc1 ; c1ccc(-c2ccc(-c3nc(-c4ccccc4)nc(-c4ccc(-c5ccc6nc(-c7cccnc7)c(-c7ccccn7)nc6c5)cc4)n3)cc2)cc1 ; c1ccc(-c2nc(-c3ccc(-c4ccc5nc(-c6cccnc6)c(-c6ccccn6)nc5c4)cc3)nc(-c3ccc4ccccc4c3)n2)cc1 ; c1ccc(-c2nc(-c3ccc(-c4ccc5ccccc5c4)cc3)nc(-c3ccc(-c4ccc5nc(-c6cccnc6)c(-c6ccccn6)nc5c4)cc3)n2)cc1]

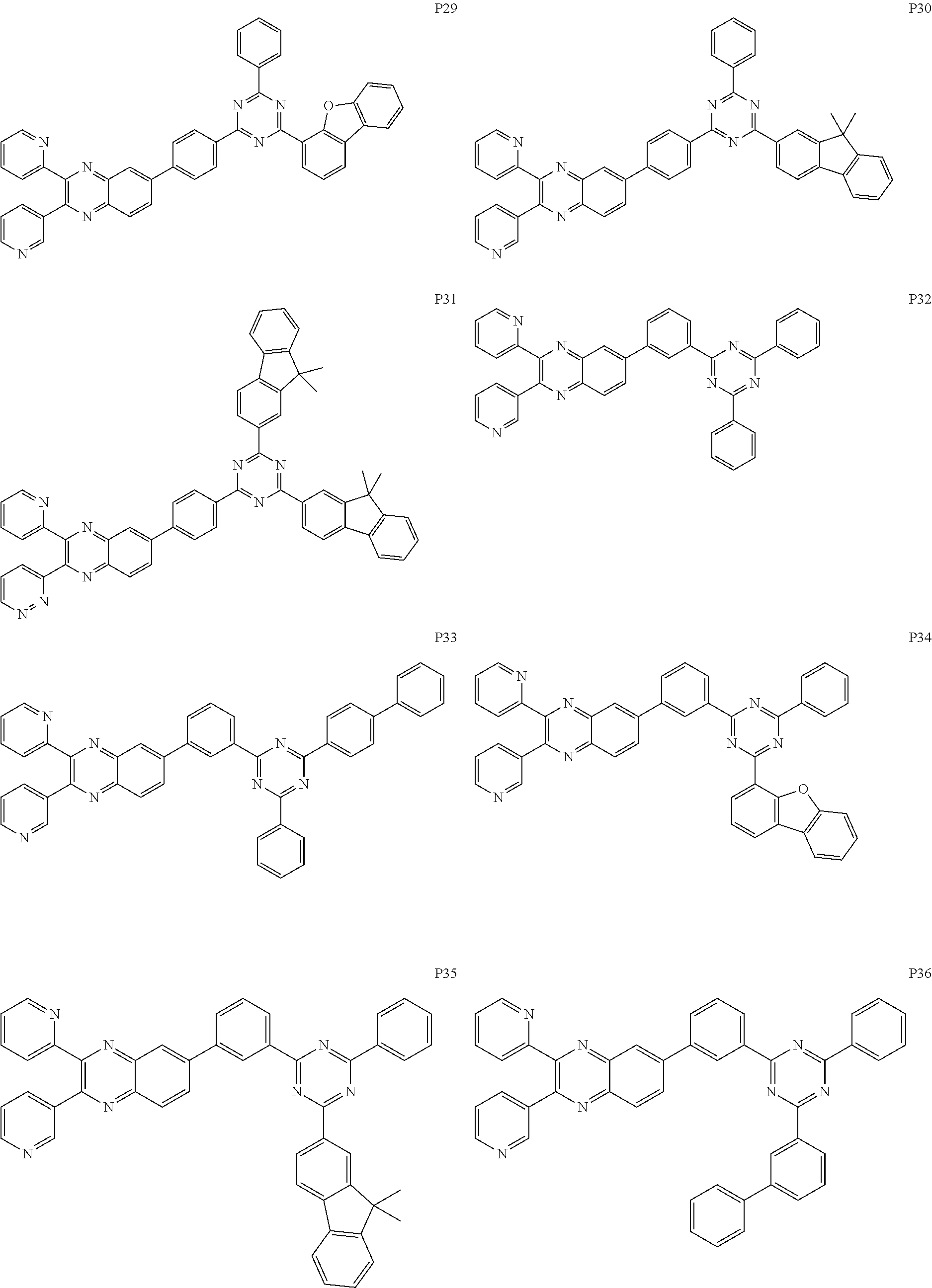

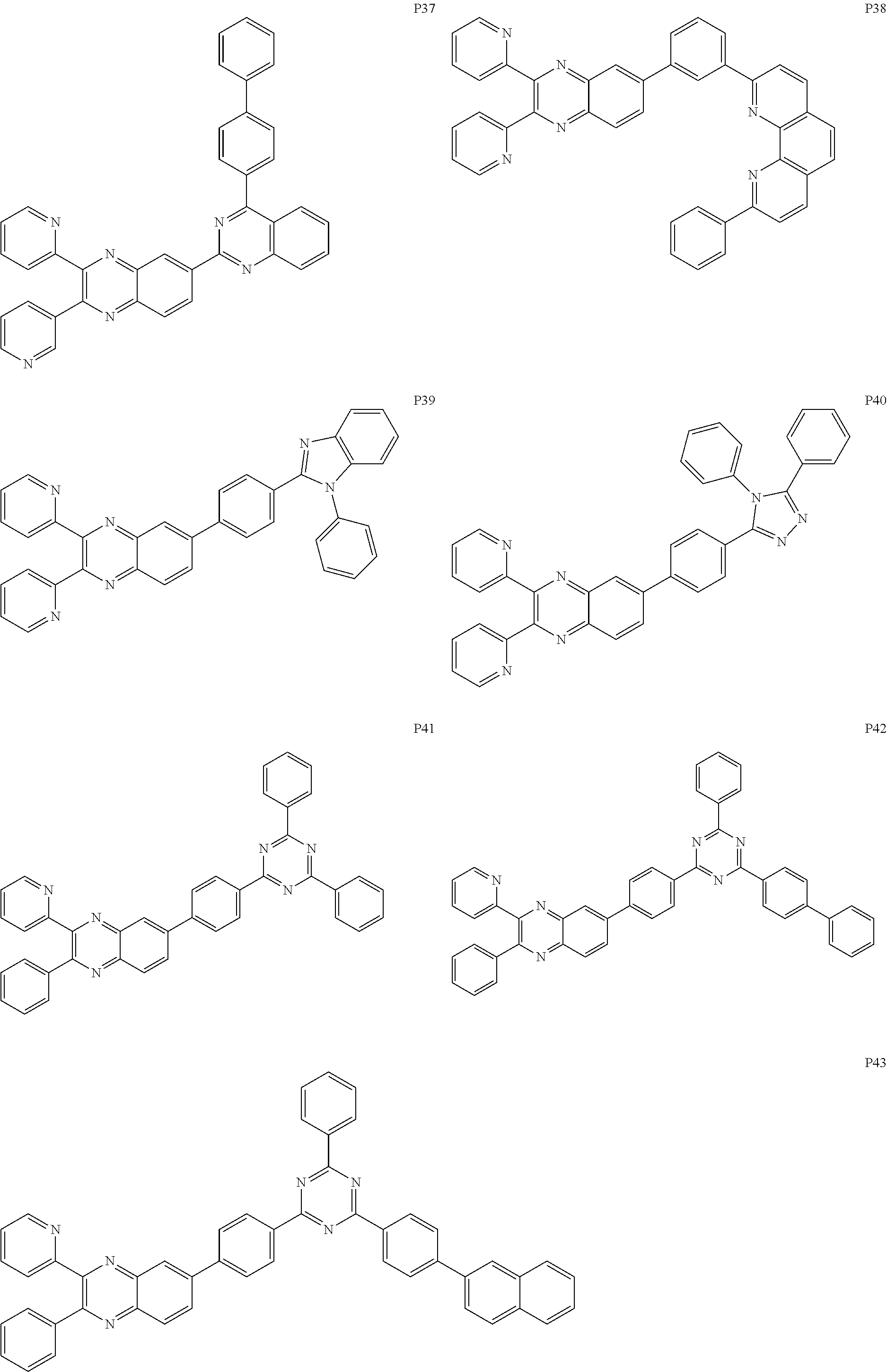

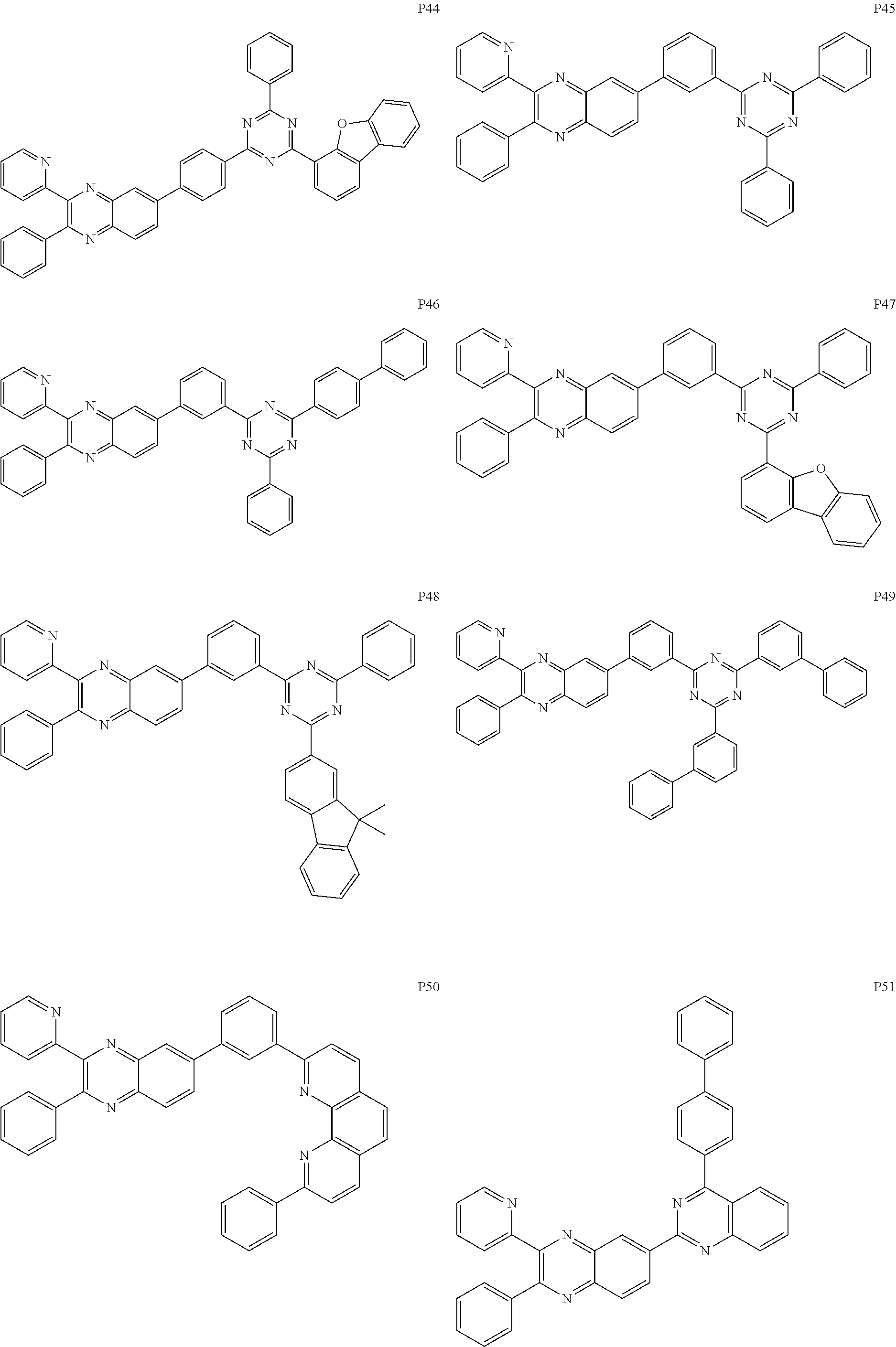

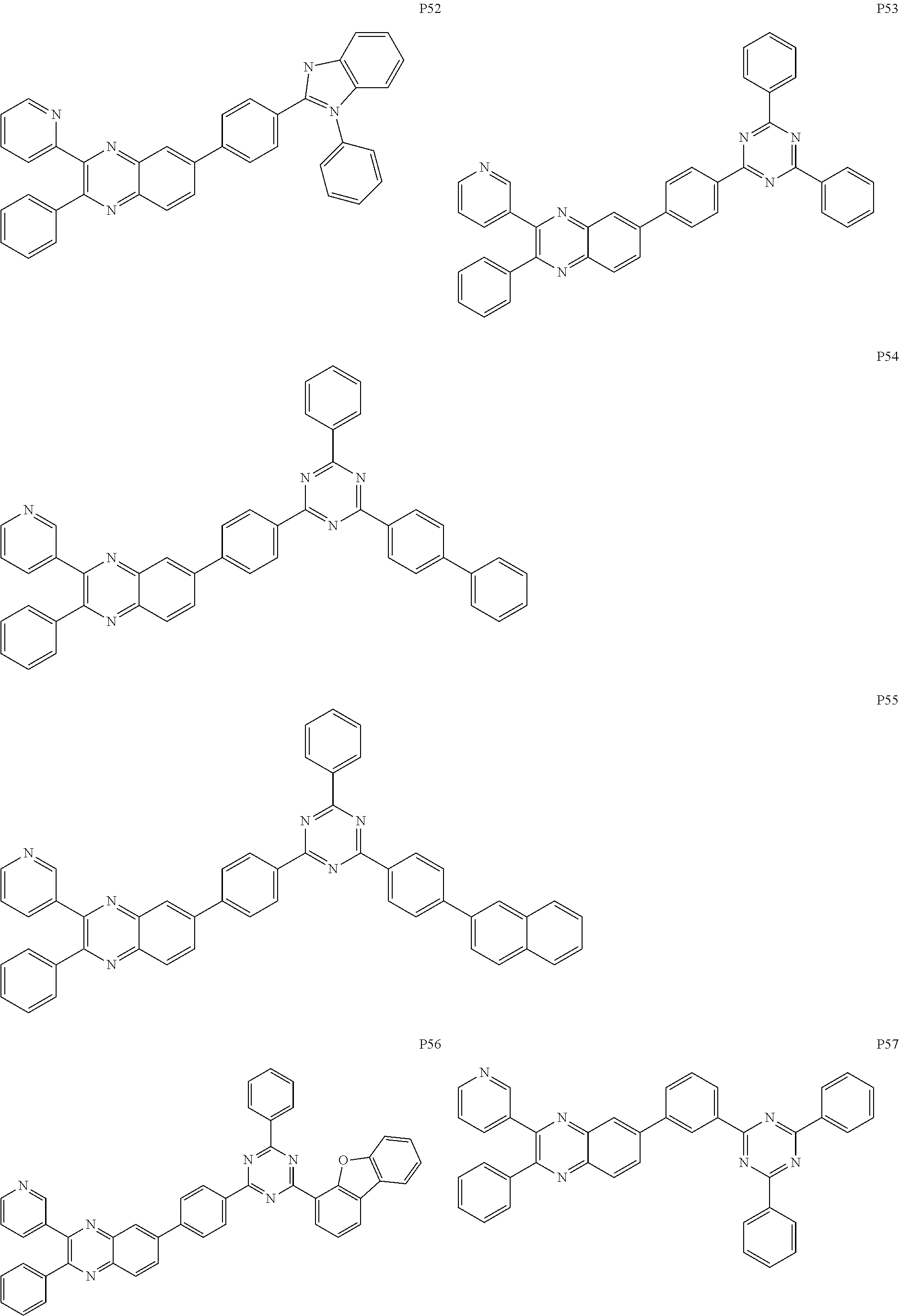

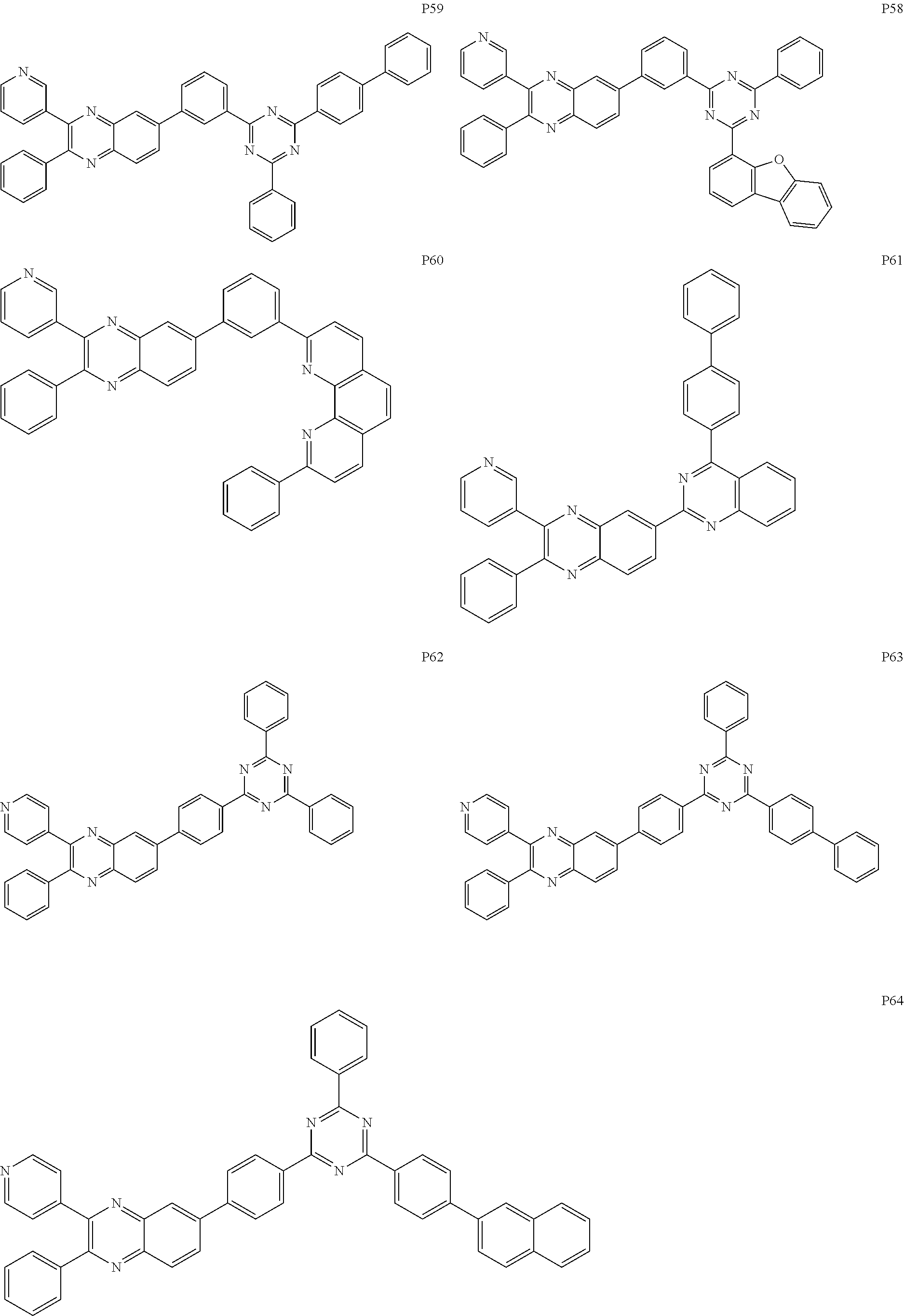

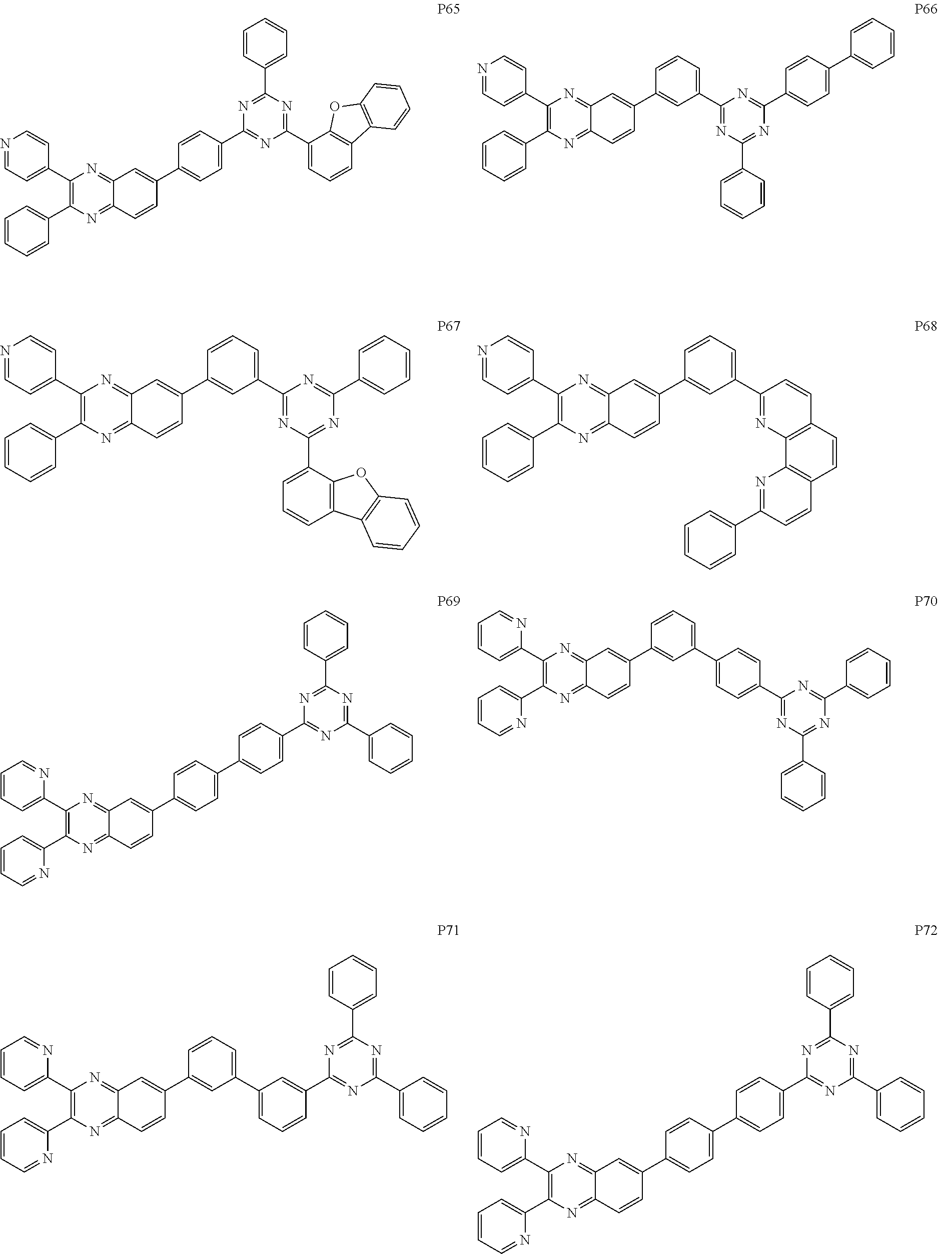

In another aspect, the present disclosure provides an electron transmission material, the electron transmission material includes the quinoxaline compound according to the first object.

In another aspect, the present disclosure provides an organic electroluminescent device, the organic electroluminescent device includes a cathode, an anode, and an organic film layer located between the cathode and the anode, where the organic film layer includes an electron transmission layer, and the electron transmission layer includes at least one of the quinoxaline compounds according to the first object.

In another aspect, the present disclosure provides a display panel, where the display panel includes the organic electroluminescent device according to the third object.

In another aspect, the present disclosure provides an organic light-emitting display device, the organic light-emitting display device includes the display panel according to the fourth object.

In another aspect, the present disclosure provides an electronic device, where the electronic device includes the display panel according to the fourth object.

Compared with the prior art, the aspects and embodiments of the present disclosure many beneficial and advantageous characteristics, some of which are described below.

At least one pyridine ring is introduced to the nitrogen-containing ring of quinoxaline of the compound of the present disclosure, which is advantageous in that, firstly, the position of N atom of the pyridine ring can adjust LUMO and HOMO energy levels of the molecule to provide a better match with the energy levels of the adjacent layer; secondly, the introduction of pyridine ring further enhances the electron transmission ability, balance electrons and holes, and improve the luminescence efficiency of the device; thirdly, the introduction of pyridine ring, especially two pyridine rings, better coordinates with metals, such as Yb, Li, etc. to achieve effective injection and transfer of electrons, lowering the operating voltage of the device, and enhancing the luminescence efficiency.

On the basis of the above, other electron-withdrawing groups are introduced into the benzene ring of quinoxaline. On one hand, by adjusting type and bonding position of the electron-withdrawing groups, it is possible to freely adjust the LUMO and HOMO energy levels of the molecule, which can be matched with the energy levels of the adjacent layers to effectively enhance the injection of electrons. On the other hand, the introduction of other electron-withdrawing groups can improve electron mobility, so as to achieve balance of the electron and hole transmission. When the introduced electron-withdrawing group has an asymmetric structure, steric hindrance can be improved. Thus, the molecule is not easy to crystallize, thermal stability of the material is improved, and therefore service life of the device is prolonged. Therefore, the compound of the present disclosure can be applied as an electron transmission material, which can reduce operating voltage of the device, improve light-emitting efficiency of the device, and prolong service life of the device. In addition, the compound of the present disclosure also has a relatively deep HOMO energy level, has a relatively good blocking capability for holes, and thus can be used as a hole blocking material.

### BRIEF DESCRIPTION OF DRAWINGS

In order to make the technical solutions according to the embodiments of the present disclosure or the prior art more apparent, the drawings to which a description of the embodiments or the prior art refers to will be introduced below in brief. Apparently, the drawings to be described below only correspond to some embodiments of the present disclosure, and those ordinarily skilled in the art can further drive from these drawings other drawings without any creative effort.

FIG. 1 is a structural schematic diagram of an organic electroluminescent device according to the present disclosure, where 1: glass substrate, 2: ITO anode, 3: hole injection layer, 4: hole transmission layer, 5: electron blocking layer, 6: light emitting layer, 7: hole blocking layer, 8: electron transmission layer, 9: cathode, and 10: covering layer.

FIG. 2 is a schematic diagram of a display device according to an embodiment of the present disclosure, where 20: mobile phone display panel, and 30: display device.

### DESCRIPTION OF EMBODIMENTS

In order to better understand the technical solutions of the present disclosure, embodiments of the present disclosure are described in detail as follows with reference to the accompanying drawings.

It should be noted that, the described embodiments are merely a part of the embodiment of the present disclosure but not all of the embodiments. Based on the embodiments in the present disclosure, all other embodiments obtained by those skilled in the art without any creative efforts shall fall into the protection scope of the present disclosure.

The terms used in the embodiments of the present disclosure are only for the purpose of describing specific examples, and are not intended to limit the present disclosure. The singular forms of “a”, “said” and “the” used in the embodiments of the present disclosure and the appended claims are also intended to include the plurality form, unless the context clearly indicates other meanings.

It will be understood that the term “and/or” as used herein is merely an associative relationship that describes associated objects, that there may be three relationships, e. g., A and/or B, which may mean that A alone is present, while both A and B are present, B alone is present, three of which exist alone. In addition, the character “/” used herein usually indicates an “or” relation between the associated objects.

### Example 1 Synthesis of Intermediate A

Using Intermediate Al as an example, bis(2-pyridyl) ethanedione (21.2 g, 0.1 mol), 4,5-dibromo-o-phenylenediamine (26.6 g, 0.1 mol) and acetic acid (200 mL) were added into a flask and reacted at 90° C. for 5 hours. After the reaction was completed and cooled to room temperature, the reaction mixture was poured into ice water, the precipitate was filtered, and the filter cake was recrystallized with ethanol, to obtain the Intermediate Al (36.7 g, yield 83%).

Table 1 shows the raw materials corresponding to the synthesis of Intermediate A. Using the same molar ratio, the synthesis methods of the remaining intermediates A2˜ to A4 are the same as those of A1.

TABLE 1

| Raw Material A | Raw Material B | Intermediate A |
|---|---|---|
| 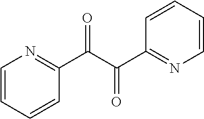 [SMILES: O=C(C(=O)c1ccccn1)c1ccccn1] | 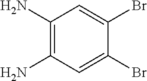 | 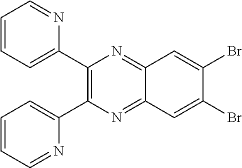 [SMILES: Brc1cc2nc(-c3ccccn3)c(-c3ccccn3)nc2cc1Br] |
| | | Intermediate A1 |
| 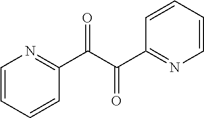 [SMILES: O=C(C(=O)c1ccccn1)c1ccccn1] | 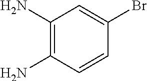 | 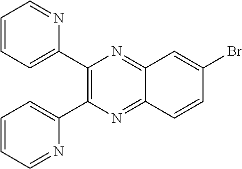 [SMILES: Brc1ccc2nc(-c3ccccn3)c(-c3ccccn3)nc2c1] |
| | | Intermediate A2 |
| 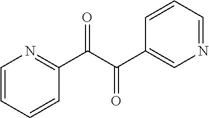 [SMILES: O=C(C(=O)c1ccccn1)c1cccnc1] | 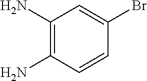 | 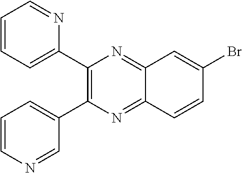 [SMILES: Brc1ccc2nc(-c3cccnc3)c(-c3ccccn3)nc2c1] |
| | | Intermediate A3 |
| 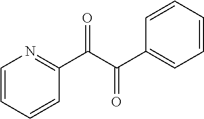 [SMILES: O=C(C(=O)c1ccccn1)c1ccccc1] | 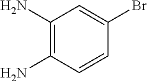 | 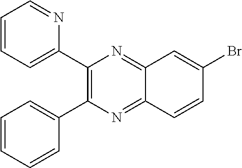 [SMILES: Brc1ccc2nc(-c3ccccc3)c(-c3ccccn3)nc2c1] |
| | | Intermediate A4 |

### Example 2 Synthesis of Intermediate B

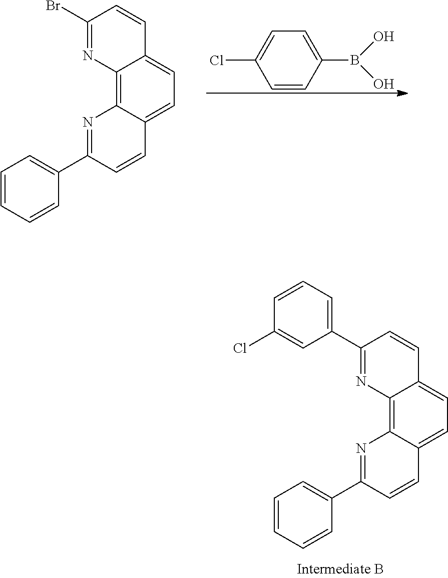 [SMILES: Brc1ccc2ccc3ccc(-c4ccccc4)nc3c2n1 ; OB(O)c1ccc(Cl)cc1 ; Clc1cccc(-c2ccc3ccc4ccc(-c5ccccc5)nc4c3n2)c1]

Under a nitrogen atmosphere, intermediate 2-phenyl-9-bromo-1,10-phenanthroline (33.5 g, 0.1 mol), 4-chlorophenylboronic acid (18.8 g, 0.12 mol), tetrakis(triphenylphosphine) palladium (5.8 g, 0.005 mol), and potassium carbonate (40.8 g, 0.295 mol) were mixed with 280 mL of toluene, 70 mL of ethanol, and 70 mL of water, and stirred at 110° C. for 12 hours. After the reaction was completed, the mixture was extracted with dichloromethane, the extract was dried over anhydrous sodium sulfate, filtered, and the filtrate was distilled under reduced pressure and purified by silica gel column, to obtain the Intermediate B (31.2 g, yield 85%).

### Example 3 Synthesis of Intermediate C

Using Intermediate C1 as an example, under a nitrogen atmosphere, 6-bromoquinoline (4.2 g, 20 mmol), bis(pinacolato) diboron (6.1 g, 24 mmol), (1,1′-bis(diphenylphosphino) ferrocene) palladium dichloride (0.44 g, 0.6 mmol), potassium acetate (3.9 g, 40 mmol) were mixed with 100 mL of tetrahydrofuran, and stirred at 100° C. for 12 hours. After the reaction was completed, the mixture was extracted with dichloromethane, the extract was dried over anhydrous sodium sulfate, filtered, and the filtrate was distilled under reduced pressure and purified by silica gel column, to obtain the Intermediate C1 (4.6 g, yield 91%).

Table 2 shows the raw materials corresponding to the synthesis of the Intermediate C. Using the same molar ratio, the synthesis methods of the remaining intermediates C2 to C6 are the same as those of C1.

TABLE 2

| Raw Material C | Raw Material D | Intermediate C |
|---|---|---|
| 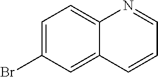 [SMILES: Brc1ccc2ncccc2c1] | 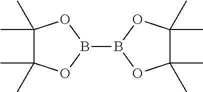 | 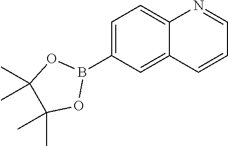 [SMILES: CC1(C)OB(c2ccc3ncccc3c2)OC1(C)C] |
| | | Intermediate C1 |
| 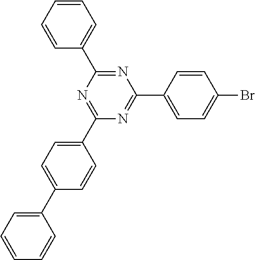 [SMILES: Brc1ccc(-c2nc(-c3ccccc3)nc(-c3ccc(-c4ccccc4)cc3)n2)cc1] | 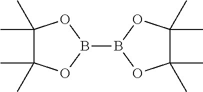 | 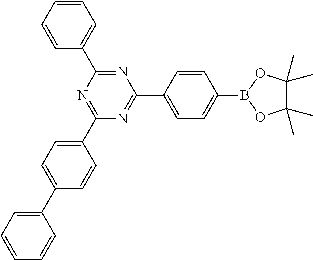 [SMILES: CC1(C)OB(c2ccc(-c3nc(-c4ccccc4)nc(-c4ccc(-c5ccccc5)cc4)n3)cc2)OC1(C)C] |
| | | Intermediate C2 |
| 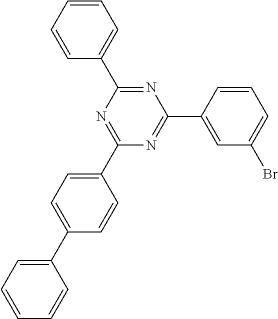 [SMILES: Brc1cccc(-c2nc(-c3ccccc3)nc(-c3ccc(-c4ccccc4)cc3)n2)c1] | 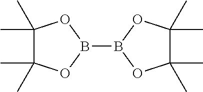 | 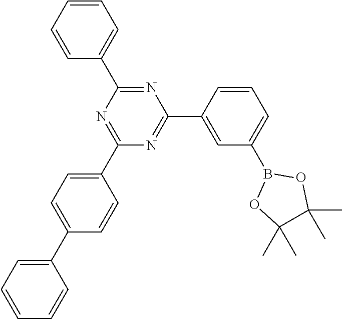 [SMILES: CC1(C)OB(c2cccc(-c3nc(-c4ccccc4)nc(-c4ccc(-c5ccccc5)cc4)n3)c2)OC1(C)C] |
| | | Intermediate C3 |
| 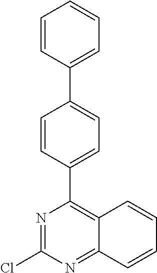 [SMILES: Clc1nc(-c2ccc(-c3ccccc3)cc2)c2ccccc2n1] | 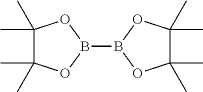 | 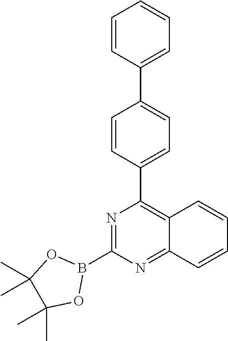 [SMILES: CC1(C)OB(c2nc(-c3ccc(-c4ccccc4)cc3)c3ccccc3n2)OC1(C)C] |
| | | Intermediate C4 |
| 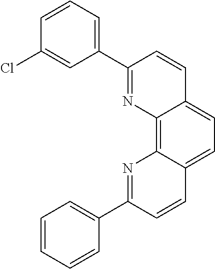 [SMILES: Clc1cccc(-c2ccc3ccc4ccc(-c5ccccc5)nc4c3n2)c1] | 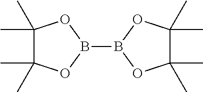 | 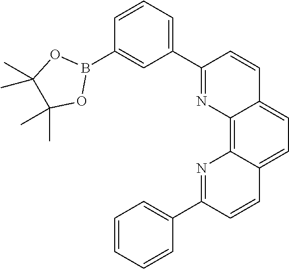 [SMILES: CC1(C)OB(c2cccc(-c3ccc4ccc5ccc(-c6ccccc6)nc5c4n3)c2)OC1(C)C] |
| Intermediate B | | Intermediate C5 |
| 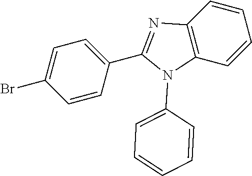 [SMILES: Brc1ccc(-c2nc3ccccc3n2-c2ccccc2)cc1] | 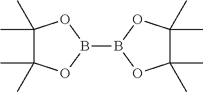 | 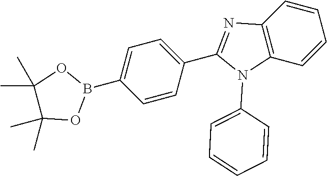 [SMILES: CC1(C)OB(c2ccc(-c3nc4ccccc4n3-c3ccccc3)cc2)OC1(C)C] |
| | | Intermediate C6 |

### Example 4 Synthesis of Compounds P

### Synthesis of Compound P01

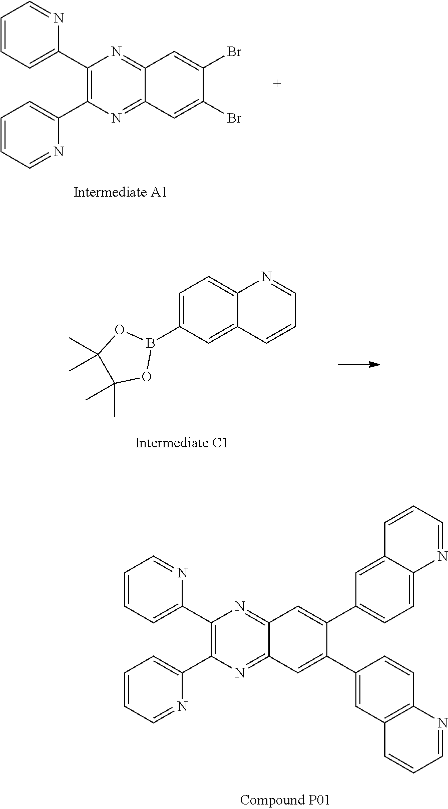 [SMILES: Brc1cc2nc(-c3ccccn3)c(-c3ccccn3)nc2cc1Br ; CC1(C)OB(c2ccc3ncccc3c2)OC1(C)C ; c1ccc(-c2nc3cc(-c4ccc5ncccc5c4)c(-c4ccc5ncccc5c4)cc3nc2-c2ccccn2)nc1]

Under nitrogen atmosphere, Intermediate Al (4.4 g, 0.01 mol), Intermediate C1 (6.4 g, 0.025 mol), tetrakis(triphenylphosphine) palladium (1.0 g, 0.9 mmol), and potassium carbonate (8.2 g, 0.059 mol) were mixed with 120 mL of toluene, 30 mL of water, and 30 mL of ethanol, and stirred at 110° C. for 12 hours. After the reaction was completed, the mixture was extracted with dichloromethane, the extract was dried over anhydrous sodium sulfate, filtered, and the filtrate was distilled under reduced pressure and purified by silica gel column, to obtain the target Compound P01 (4.5 g, yield 83%).

Compound P06, Compound P17, Compound P21, Compound P22, Compound P23, Compound P26, Compound P33, Compound P38, Compound P42, Compound P46, and Compound P50 were prepared by the same synthesis method as for P01, expect that: Compound P06 was prepared with a molar ratio of Intermediate A1:Intermediate C1:tetrakis(triphenylphosphine) palladium:potassium carbonate=1:2.5:0.09:5.9;

Compound P17, Compound P21, Compound P22, Compound P23, Compound P26, Compound P33, Compound P38, Compound P42, Compound P46, and Compound P50 were prepared with a molar ratio of Intermediate A:Intermediate C:tetrakis(triphenylphosphine) palladium:potassium carbonate=1:1.5:0.03:2.95.

TABLE 3

| Intermediate A | Intermediate C | Compound P |
|---|---|---|
| 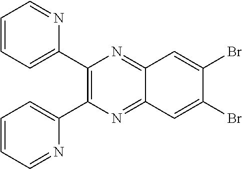 [SMILES: Brc1cc2nc(-c3ccccn3)c(-c3ccccn3)nc2cc1Br] | 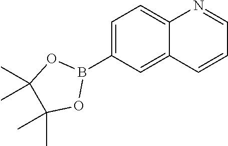 [SMILES: CC1(C)OB(c2ccc3ncccc3c2)OC1(C)C] | 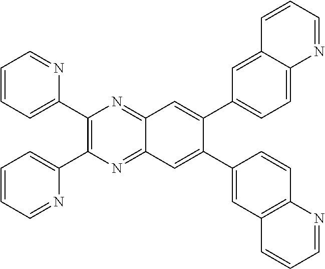 [SMILES: c1ccc(-c2nc3cc(-c4ccc5ncccc5c4)c(-c4ccc5ncccc5c4)cc3nc2-c2ccccn2)nc1] |
| Intermediate A1 | Intermediate CI | Compound P91 |
| 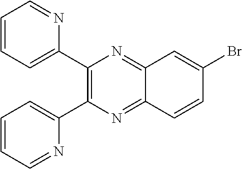 [SMILES: Brc1ccc2nc(-c3ccccn3)c(-c3ccccn3)nc2c1] | 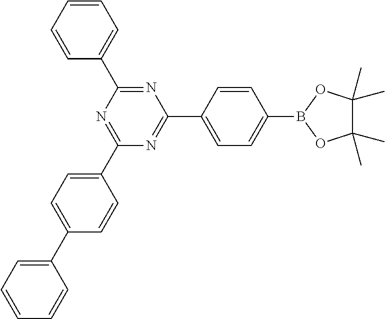 [SMILES: CC1(C)OB(c2ccc(-c3nc(-c4ccccc4)nc(-c4ccc(-c5ccccc5)cc4)n3)cc2)OC1(C)C] | 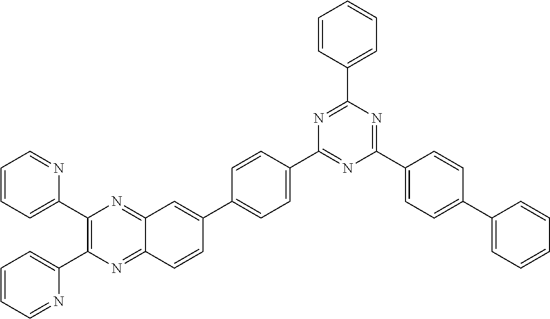 [SMILES: c1ccc(-c2ccc(-c3nc(-c4ccccc4)nc(-c4ccc(-c5ccc6nc(-c7ccccn7)c(-c7ccccn7)nc6c5)cc4)n3)cc2)cc1] |
| Intermediate A2 | Intermediate C2 | Compound PO6 |
| 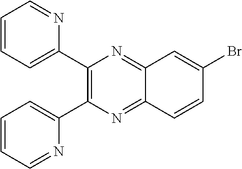 [SMILES: Brc1ccc2nc(-c3ccccn3)c(-c3ccccn3)nc2c1] | 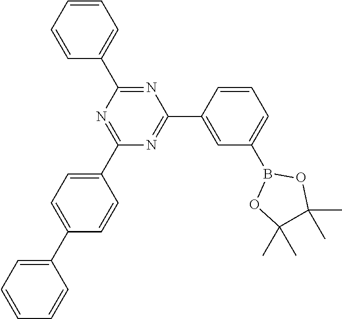 [SMILES: CC1(C)OB(c2cccc(-c3nc(-c4ccccc4)nc(-c4ccc(-c5ccccc5)cc4)n3)c2)OC1(C)C] | 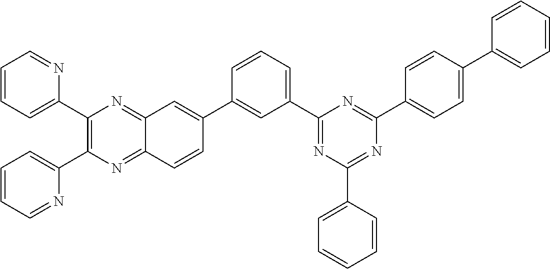 [SMILES: c1ccc(-c2ccc(-c3nc(-c4ccccc4)nc(-c4cccc(-c5ccc6nc(-c7ccccn7)c(-c7ccccn7)nc6c5)c4)n3)cc2)cc1] |
| Intermediate A2 | Intermediate C3 | Compound P17 |
| 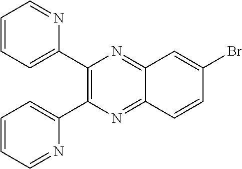 [SMILES: Brc1ccc2nc(-c3ccccn3)c(-c3ccccn3)nc2c1] | 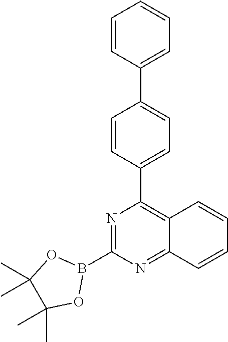 [SMILES: CC1(C)OB(c2nc(-c3ccc(-c4ccccc4)cc3)c3ccccc3n2)OC1(C)C] | 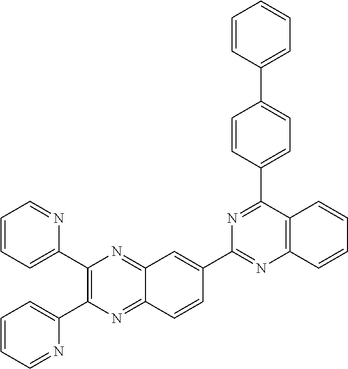 [SMILES: c1ccc(-c2ccc(-c3nc(-c4ccc5nc(-c6ccccn6)c(-c6ccccn6)nc5c4)nc4ccccc34)cc2)cc1] |
| Intermediate A2 | Intermediate C4 | Compound P21 |
| 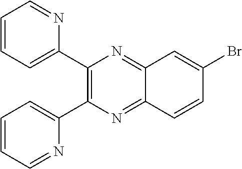 [SMILES: Brc1ccc2nc(-c3ccccn3)c(-c3ccccn3)nc2c1] | 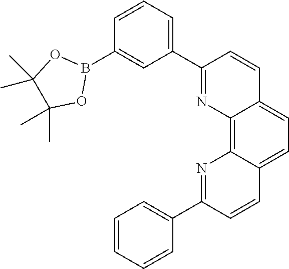 [SMILES: CC1(C)OB(c2cccc(-c3ccc4ccc5ccc(-c6ccccc6)nc5c4n3)c2)OC1(C)C] | 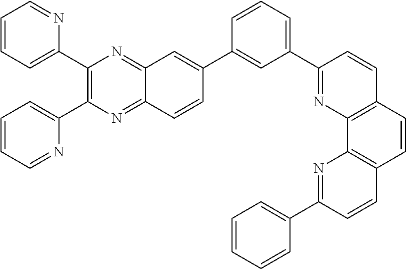 [SMILES: c1ccc(-c2ccc3ccc4ccc(-c5cccc(-c6ccc7nc(-c8ccccn8)c(-c8ccccn8)nc7c6)c5)nc4c3n2)cc1] |
| Intermediate A2 | Intermediate C5 | Compound P22 |
| 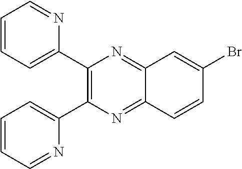 [SMILES: Brc1ccc2nc(-c3ccccn3)c(-c3ccccn3)nc2c1] | 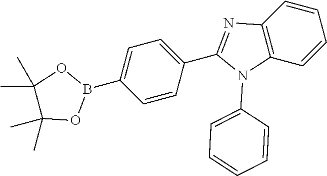 [SMILES: CC1(C)OB(c2ccc(-c3nc4ccccc4n3-c3ccccc3)cc2)OC1(C)C] | 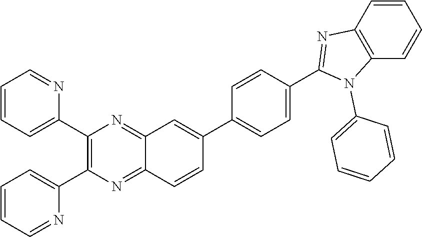 [SMILES: c1ccc(-n2c(-c3ccc(-c4ccc5nc(-c6ccccn6)c(-c6ccccn6)nc5c4)cc3)nc3ccccc32)cc1] |
| Intermediate A2 | Intermediate C6 | Compound P23 |
| 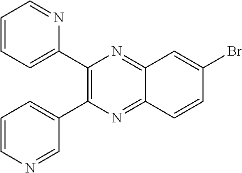 [SMILES: Brc1ccc2nc(-c3cccnc3)c(-c3ccccn3)nc2c1] | 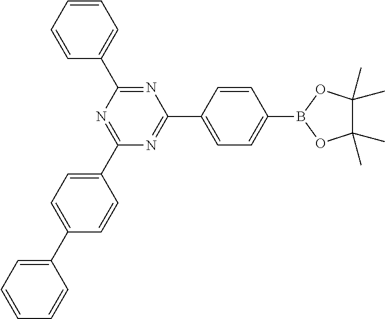 [SMILES: CC1(C)OB(c2ccc(-c3nc(-c4ccccc4)nc(-c4ccc(-c5ccccc5)cc4)n3)cc2)OC1(C)C] | 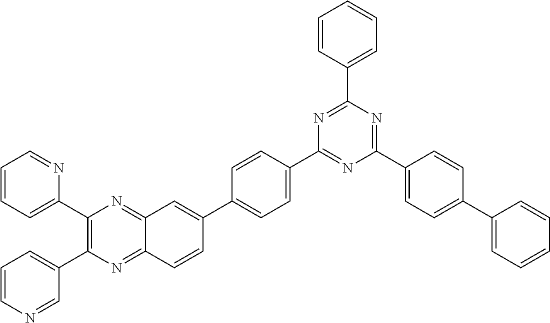 [SMILES: c1ccc(-c2ccc(-c3nc(-c4ccccc4)nc(-c4ccc(-c5ccc6nc(-c7cccnc7)c(-c7ccccn7)nc6c5)cc4)n3)cc2)cc1] |
| Intermediate A3 | Intermediate C2 | Compound P26 |
| 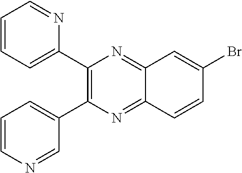 [SMILES: Brc1ccc2nc(-c3cccnc3)c(-c3ccccn3)nc2c1] | 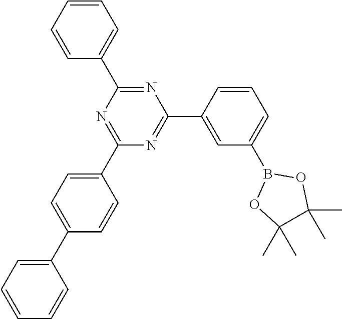 [SMILES: CC1(C)OB(c2cccc(-c3nc(-c4ccccc4)nc(-c4ccc(-c5ccccc5)cc4)n3)c2)OC1(C)C] | 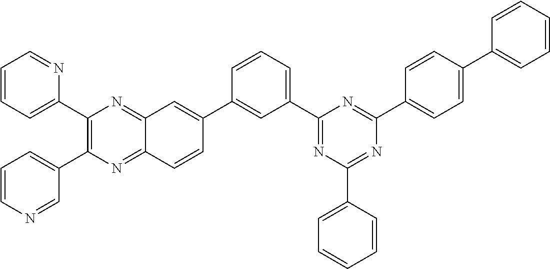 [SMILES: c1ccc(-c2ccc(-c3nc(-c4ccccc4)nc(-c4cccc(-c5ccc6nc(-c7cccnc7)c(-c7ccccn7)nc6c5)c4)n3)cc2)cc1] |
| Intermediate A3 | Intermediate C3 | Compound P33 |
| 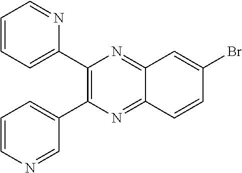 [SMILES: Brc1ccc2nc(-c3cccnc3)c(-c3ccccn3)nc2c1] | 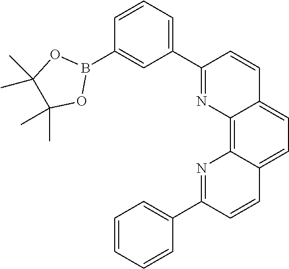 [SMILES: CC1(C)OB(c2cccc(-c3ccc4ccc5ccc(-c6ccccc6)nc5c4n3)c2)OC1(C)C] | 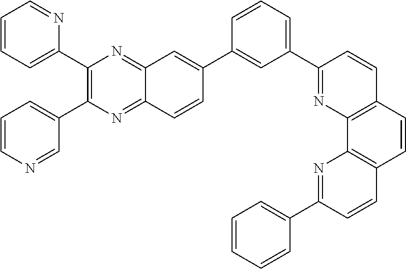 [SMILES: c1ccc(-c2ccc3ccc4ccc(-c5cccc(-c6ccc7nc(-c8cccnc8)c(-c8ccccn8)nc7c6)c5)nc4c3n2)cc1] |
| Intermediate A3 | Intermediate C5 | Compound P38 |
| 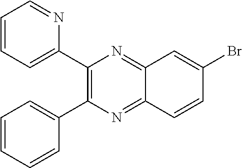 [SMILES: Brc1ccc2nc(-c3ccccc3)c(-c3ccccn3)nc2c1] | 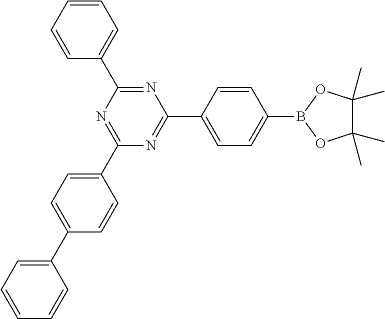 [SMILES: CC1(C)OB(c2ccc(-c3nc(-c4ccccc4)nc(-c4ccc(-c5ccccc5)cc4)n3)cc2)OC1(C)C] | 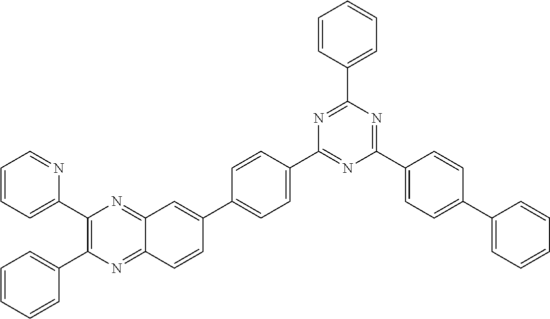 [SMILES: c1ccc(-c2ccc(-c3nc(-c4ccccc4)nc(-c4ccc(-c5ccc6nc(-c7ccccc7)c(-c7ccccn7)nc6c5)cc4)n3)cc2)cc1] |
| Intermediate A4 | Intermediate C2 | Compound P42 |
| 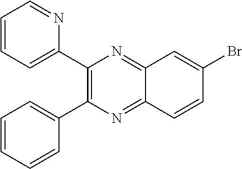 [SMILES: Brc1ccc2nc(-c3ccccc3)c(-c3ccccn3)nc2c1] | 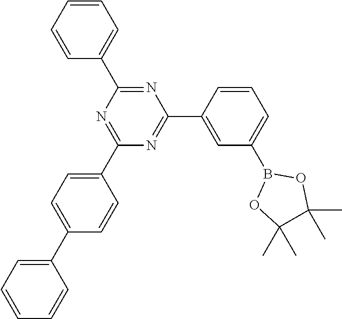 [SMILES: CC1(C)OB(c2cccc(-c3nc(-c4ccccc4)nc(-c4ccc(-c5ccccc5)cc4)n3)c2)OC1(C)C] | 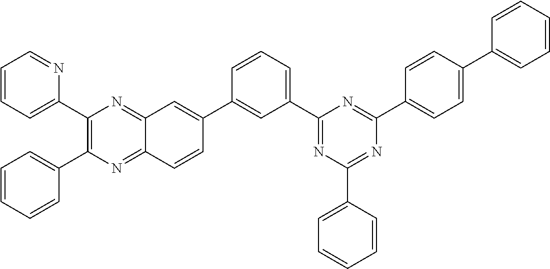 [SMILES: c1ccc(-c2ccc(-c3nc(-c4ccccc4)nc(-c4cccc(-c5ccc6nc(-c7ccccc7)c(-c7ccccn7)nc6c5)c4)n3)cc2)cc1] |
| Intermediate A4 | Intermediate C3 | Compound P46 |
| 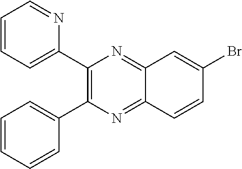 [SMILES: Brc1ccc2nc(-c3ccccc3)c(-c3ccccn3)nc2c1] | 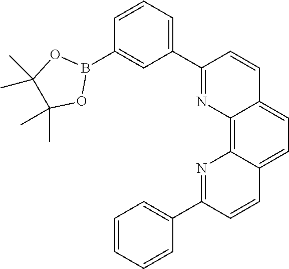 [SMILES: CC1(C)OB(c2cccc(-c3ccc4ccc5ccc(-c6ccccc6)nc5c4n3)c2)OC1(C)C] | 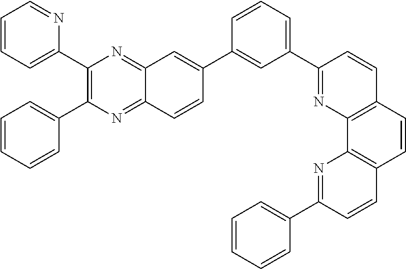 [SMILES: c1ccc(-c2ccc3ccc4ccc(-c5cccc(-c6ccc7nc(-c8ccccc8)c(-c8ccccn8)nc7c6)c5)nc4c3n2)cc1] |
| Intermediate A4 | Intermediate C5 | Compound P50 |

The compounds in the above Examples can be determined by matrix-assisted laser desorption ionization time-of-flight mass spectrometry (abbreviated as MALDI-TOF MS) and elemental analysis, and the results are shown in Table 4 below.

TABLE 4

| | Theoretical Value | Measured Value | | |
|---|---|---|---|---|
| | of MALDI-TOF | of MALDI-TOF | Theoretical Value of | Measured Value of |
| Compound | MS | MS | Elemental Analysis | Elemental Analysis |
| Intermediate A1 | 439.93 | 43 [symbol] .81 | C [symbol] 48.90 [symbol] H [symbol] 2.28 [symbol] Br [symbol] | C [symbol] 48.91 [symbol] H [symbol] 2.27 [symbol] Br [symbol] |
| | | | 36.15 [symbol] N [symbol] 12.67 | 36.13 [symbol] N [symbol] 12.69 |
| Intermediate A2 | 362.02 | 362.15 | C [symbol] 59.52 [symbol] H [symbol] 3.05 [symbol] Br [symbol] | C [symbol] 59. [symbol] H [symbol] 3.03 [symbol] Br [symbol] |
| | | | 22.00 [symbol] N [symbol] 15.43 | 22.0 [symbol] N [symbol] 15.41 |
| Intermediate A3 | 362.02 | 362.13 | C [symbol] 59.52 [symbol] H [symbol] .0 [symbol] Br [symbol] | C [symbol] 59. [symbol] H [symbol] 3.03 [symbol] Br [symbol] |
| | | | 22.00 [symbol] N [symbol] 15.43 | 22.03 [symbol] N [symbol] 15.41 |
| Intermediate A4 | 361.02 | 361.08 | C [symbol] 63.00 [symbol] H [symbol] 3.34 [symbol] Br [symbol] | C [symbol] 63.02 [symbol] H [symbol] 3.35 [symbol] Br [symbol] |
| | | | 22.06 [symbol] N [symbol] 11.60 | 22.02 [symbol] N [symbol] 11.61 |
| Intermediate B | 366.09 | 366.15 | C [symbol] 78. [symbol] H [symbol] 4.12 [symbol] C [symbol] | C [symbol] 78.57 [symbol] H [symbol] 4.11 [symbol] C [symbol] |
| | | | 9.66 [symbol] N [symbol] 7.64 | 9.69 [symbol] N [symbol] 7.63 |
| Intermediate C1 | 2 [symbol] .14 | 255.26 | C [symbol] 70.62 [symbol] H [symbol] 7.11 [symbol] B [symbol] | C [symbol] 70.61 [symbol] H [symbol] 7.10 [symbol] B [symbol] |
| | | | 4.24 [symbol] N [symbol] 5.49 [symbol] O [symbol] 12.54 | 4.22 [symbol] N [symbol] 5.48 [symbol] O [symbol] 12.59 |
| Intermediate C2 | 511.24 | 511.32 | C [symbol] 77. [symbol] 0 [symbol] H [symbol] 5.91 [symbol] B [symbol] | C [symbol] 77.51 [symbol] H [symbol] 5.90 [symbol] B [symbol] |
| | | | 2.11 [symbol] N [symbol] .22 [symbol] O [symbol] 6.26 | 2.09 [symbol] N [symbol] 8.23 [symbol] O [symbol] 6.27 |
| Intermediate C3 | 511.24 | 511.35 | C [symbol] 77.50 [symbol] H [symbol] 5.91 [symbol] B [symbol] | C [symbol] 77.51 [symbol] H [symbol] 5.92 [symbol] B [symbol] |
| | | | 2.11 [symbol] N [symbol] 8.22 [symbol] O [symbol] 6.26 | 2.09 [symbol] N [symbol] 8.21 [symbol] O [symbol] 6.27 |
| Intermediate C4 | 40 [symbol] .20 | 40 [symbol] .32 | C [symbol] 76.48 [symbol] H [symbol] 6.17 [symbol] B [symbol] | C [symbol] 76.49 [symbol] H [symbol] 6.18 [symbol] B [symbol] |
| | | | 2.65 [symbol] N [symbol] 6.86 [symbol] O [symbol] 7.84 | 2.61 [symbol] N [symbol] 6.87 [symbol] O [symbol] 7.85 |
| Intermediate C5 | 45 [symbol] .22 | 458.3 [symbol] | C [symbol] 78.61 [symbol] H [symbol] 5.94 [symbol] B [symbol] | C [symbol] 78.60 [symbol] H [symbol] 5.93 [symbol] B [symbol] |
| | | | 2.36 [symbol] N [symbol] 6.11 [symbol] O [symbol] 6. [symbol] | 2.37 [symbol] N [symbol] 6.13 [symbol] O [symbol] 6.97 |
| Intermediate C6 | 396.20 | 396.28 | C [symbol] 75.77 [symbol] H [symbol] 6.36 [symbol] B [symbol] | C [symbol] 75.78 [symbol] H [symbol] 6.37 [symbol] B [symbol] |
| | | | 2.73 [symbol] N [symbol] 7.07 [symbol] O [symbol] 8.07 | 2.72 [symbol] N [symbol] 7.08 [symbol] O [symbol] 8.05 |
| Compound P01 | 538.19 | 538.23 | C [symbol] 80.28 [symbol] H [symbol] 4.12 [symbol] N [symbol] | C [symbol] 80.27 [symbol] H [symbol] 4.14 [symbol] N [symbol] |
| | | | 15.60 | 15.59 |
| Compound P06 | 667.25 | 667.13 | C [symbol] 80.94 [symbol] H [symbol] 4.38 [symbol] N [symbol] | C [symbol] 80.93 [symbol] H [symbol] 4.37 [symbol] N [symbol] |
| | | | 14.68 | 14.70 |
| Compound P17 | 667.25 | 667.28 | C [symbol] 80.94 [symbol] H [symbol] 4.38 [symbol] N [symbol] | C [symbol] 80.93 [symbol] H [symbol] 4.36 [symbol] N [symbol] |
| | | | 14.68 | 14.71 |
| Compound P21 | 564.21 | 564.35 | C [symbol] 80.83 [symbol] H [symbol] 4.28 [symbol] N [symbol] | C [symbol] 80.84 [symbol] H [symbol] 4.26 [symbol] N [symbol] |
| | | | 14.88 | 14.90 |
| Compound P22 | 614.22 | 614.34 | C [symbol] 82.06 [symbol] H [symbol] 4.26 [symbol] N [symbol] | C [symbol] 82.07 [symbol] H [symbol] 4.27 [symbol] N [symbol] |
| | | | 13.67 | 13.66 |
| Compound P23 | [symbol] 2.21 | 552.34 | C [symbol] 80.42 [symbol] H [symbol] 4.38 [symbol] N [symbol] | C [symbol] 80.41 [symbol] H [symbol] 4.37 [symbol] N [symbol] |
| | | | 15.21 | 15.22 |
| Compound P26 | 667.25 | 667.31 | C [symbol] 80.94 [symbol] H [symbol] 4.38 [symbol] N [symbol] | C [symbol] 80.96 [symbol] H [symbol] 4.37 [symbol] N [symbol] |
| | | | 14.68 | 14.67 |
| Compound P33 | 667.25 | 667.35 | C [symbol] 80.94 [symbol] H [symbol] 4.38 [symbol] N [symbol] | C [symbol] 80.95 [symbol] H [symbol] 4.39 [symbol] N [symbol] |
| | | | 14.68 | 14.66 |
| Compound P38 | 614.22 | 614.29 | C [symbol] 2.06 [symbol] H [symbol] 4.26 [symbol] N [symbol] | C [symbol] 2.05 [symbol] H [symbol] 4.24 [symbol] N [symbol] |
| | | | 13.67 | 13.71 |
| Compound P42 | 666.25 | 666.36 | C [symbol] 82.86 [symbol] H [symbol] 4.54 [symbol] N [symbol] | C [symbol] 2.87 [symbol] H [symbol] 4.52 [symbol] N [symbol] |
| | | | 12.60 | 12.61 |
| Compound P46 | 666.25 | 666.32 | C [symbol] 82.86 [symbol] H [symbol] 4.54 [symbol] N [symbol] | C [symbol] 82.87 [symbol] H [symbol] 4.55 [symbol] N [symbol] |
| | | | 12.60 | 12.58 |
| Compound P50 | 613.23 | 613.14 | C [symbol] 84.15 [symbol] H [symbol] 4.43 [symbol] N [symbol] | C [symbol] 84.16 [symbol] H [symbol] 4.44 [symbol] N [symbol] |
| | | | 11.41 | 11.40 |

[symbol]

indicates data missing or illegible when filed

### Simulated Calculation of Compound Energy Levels

Using density functional theory (DFT), for the organic compounds provided by Examples of the present disclosure, the distribution conditions and energy levels of HOMO and LUMO of the molecular front orbit were optimized and calculated by the Gaussian09 package (Gaussian Inc.) at the calculation level of B3LYP/6-31G(d). Meanwhile, the singlet energy level ES and the triplet energy level ET of the compound molecules were analogously calculated based on the time density functional theory (TDFT). The results are shown in Table 5.

TABLE 5

| | | HOMO | LUMO | E [symbol] | E [symbol] |
|---|---|---|---|---|---|
| Example | Compound | (eV) | (eV) | (eV) | (eV) |
| Example 1 | Compound P01 | −6.00 | −2.10 | 3.40 | 2.51 |
| Example 2 | Compound P06 | −6.06 | −2.16 | 3.39 | 2.49 |
| Example 3 | Compound P17 | −6.05 | −2.00 | 3.43 | 2.55 |
| Example 4 | Compound P21 | −5.98 | −2.07 | 3.34 | 2.43 |
| Example 5 | Compound P22 | −5.77 | −1.89 | 3.44 | 2.48 |
| Example 6 | Compound P23 | −5.62 | −2.01 | 3.25 | 2.49 |
| Example 7 | Compound P26 | −6.14 | −2.26 | 3.35 | 2.47 |
| Example 8 | Compound P33 | −6.14 | −2.12 | 3.39 | 2.52 |
| Example 9 | Compound P38 | −5.67 | −2.08 | 3.29 | 2.46 |
| Example 10 | Compound P42 | −6.00 | −2.15 | 3.39 | 2.47 |
| Example 11 | Compound P46 | −6.00 | −1.99 | 3.43 | 2.53 |
| Example 12 | Compound P50 | −5.64 | −1.94 | 3.38 | 2.46 |

[symbol]

indicates data missing or illegible when filed

It can be seen from Table 5 that the LUMO energy level of the compounds of the present disclosure are all relatively deep, which can reduce the electron injection barrier, thus achieve effective injection of electrons, and reduce operating voltage of the organic electroluminescent device. Meanwhile, their HOMO energy levels are also all relatively deep, which can effectively block holes, thus improve light-emitting efficiency of the device.

### Device Examples

### Application Example 1

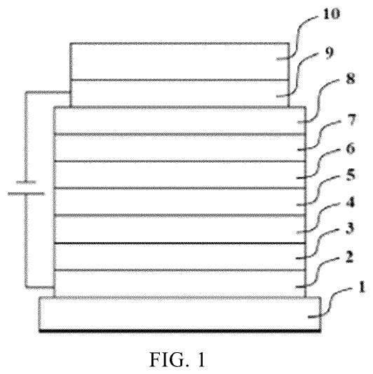

This Application Example provides an organic electroluminescent device. FIG. 1 is a schematic structural diagram of the organic electroluminescent device provided by the present disclosure, including a glass substrate 1, an anode 2, a hole injection layer 3, a first hole transmission layer 4, a second hole transmission layer 5, a light-emitting layer 6, a hole blocking layer 7, a hole transmission layer 8, a cathode 9, and a covering layer 10, which are sequentially stacked.

The materials of the hole injection layer, the hole transmission layer, and the electron blocking layer can be selected from, but not limited to, 2,2′-dimethyl-N, N′-di-1-naphthyl-N,N′-diphenyl [1,1′-biphenyl]-4,4′-diamine (α-NPD), 4,4′, 4″-tris(carbazol-9-yl) triphenylamine (TCTA), 1,3-bis(N-dicarbazolyl) benzene (mCP), 4,4′-bis(N-carbazolyl)-1,1′-biphenyl (CBP), 3,3′-bis(N-carbazolyl)-1,1′-biphenyl (mCBP), 2,3,6,7,10,11-hexacyano-1,4,5,8,9,12-hexaazatriphenylene (HATCN), 4,4′-cyclohexyl di[N,N-bis(4-methylphenyl) aniline (TAPC), N,N′-bis(1-naphthalenyl)-N,N′-diphenyl-(1,1′-biphenyl)-4,4′-diamine (α-NPB), N, N′-bis(naphthalene-2-yl)-N,N′-bis(phenyl) benzidine (NPB), poly(3,4-ethylene dioxythiophene) -poly(styrene sulfonate) (PEDOT:PSS), polyvinylcarbazole (PVK), 9-phenyl-3,9-bicarbazole (CCP), molybdenum trioxide (MoO₃), or the like.

The materials of the hole blocking layer, the electron transmission layer, and the electron injection layer can be selected from, but not limited to, 2, 8-bis(diphenyl phosphoryl) dibenzothiophene (PPT), TSPO1,1,3,5-tris(1-phenyl-1H-benzimidazol-2-yl) benzene (TPBi), 2,8-bis(diphenyl phosphoryl) dibenzofuran (PPF), bis[2-(diphenylphosphino) phenyl]ether (DPEPO), lithium fluoride (LiF), 4,6-bis(3,5-di(pyridin-3-yl) phenyl)-2-methyl pyrimidine (B3PYMPM), 4,7-diphenyl-1,10-phenanthroline (Bphen), 1,3,5-tris[(pyridin-3-yl)-3-phenyl]benzene (TmPyBP), tris[2,4,6-trimethyl-3-(pyridin-3-yl) phenyl]borane (3TPYMB), 1,3-bis(3,5-di(pyridin-3-yl) phenyl) benzene (B3PYPB), 1,3-bis[3,5-di(pyridin-3-yl) phenyl] benzene (BMPYPHB), 2,4,6-tris(biphenyl-3-yl)-1,35-triazine (T2T), diphenyl bis[4-(pyridin-3-yl) phenyl]silane (DPPS), cesium carbonate (Cs₂O₃), bis(2-methyl-8-quinolinolato-N1,O8)-(1,1′-biphenyl-4-olato) aluminum (BAlq), 8-hydroxyquinolinolato-lithium (Liq), tris(8-hydroxyquinoline) aluminum (Alq3), or the like.

In one embodiment of the organic light-emitting display device provided by the present disclosure, the light-emitting layer includes a host material and a guest material, where the host material is selected from any one or more of 2,8-bis(diphenyl phosphoryl) dibenzothiophene, 4,4′-bis(9-carbazole) biphenyl, 3,3′-bis(N-carbazolyl)-1,1′-biphenyl, 2,8-bis(diphenyl phosphoryl) dibenzofuran, bis(4-(9H-carbazolyl-9-yl) phenyl) diphenyl silane, 9-(4-tert-butyl phenyl)-3,6-bis(triphenyl silyl)-9h-carbazole, bis[2-(diphenyl phosphino)phenyl]ether oxide, 1,3-bis[3,5-di(pyridin-3-yl) phenyl]benzene, 4,6-bis(3,5-di(pyridin-3-yl)phenyl)-2-methyl pyrimidine, 9-(3-(9H-carbazolyl-9-yl) phenyl)-9H-carbazole-3-cyano, 9-phenyl-9-[4-(triphenyl silyl) phenyl]-9H-fluorene, 1,3,5-tris(1-phenyl-1H-benzimidazol-2-yl) benzene, diphenyl [4-(triphenyl silyl) phenyl]phosphine oxide, 4,4′,4″-tris(carbazol-9-yl) triphenylamine, 2,6-dicarbazole-1,5-pyridine, polyvinyl carbazole, and polyfluorene, and the guest material can be selected from one or more of fluorescent material, phosphorescent material or thermally activated delayed fluorescent material, and aggregation-induced luminescent material.

In the display panel provided by the present disclosure, the anode of the organic light-emitting device can comprise a metal, for example, copper, gold, silver, iron, chromium, nickel, manganese, palladium, platinum, and alloys thereof. The anode material can also be selected from metal oxides such as indium oxide, zinc oxide, indium tin oxide (ITO), indium zinc oxide (IZO), etc.; the anode material can also be selected from conductive polymers such as polyaniline, polypyrrole, poly(3-methylthiophene), etc. In addition to the anode material mentioned above, the anode also comprise any suitable materials known in the related art and combinations thereof, as long as the material of the anode is conductive to injecting holes.

In the display panel provided by the present disclosure, the cathode of the organic light-emitting device can be made of metal, such as aluminum, magnesium, silver, indium, tin, titanium, and alloys thereof. The cathode also comprise multiple-layer metal material, such as LiF/Al, LiO₂/Al, BaF₂/Al, and the like. In addition to the cathode materials listed above, the cathode can comprise a material selected from any materials that are conductive to electron injection, or combinations thereof, including the materials known in the related art that are suitable as the material of the cathode.

The organic light-emitting device can be manufactured according to methods well known in the art, which will not be described in detail herein. In the present disclosure, the organic light-emitting device can be manufactured as follows: An anode is formed on a transparent or opaque smooth substrate, an organic thin layer is formed on the anode, and a cathode is formed on the organic thin layer. The organic thin layer can be formed by a known method such as vapor deposition, sputtering, spin coating, dipping, ion plating, and the like.

### Application Example 1

An application example of the present disclosure provides an organic electroluminescent device, and the specific preparation steps thereof are as follows:
1) a glass substrate with anode indium tin oxide (ITO) with a thickness of 150 nm was ultrasonicated in isopropanol and deionized water for 30 minutes, respectively, then exposed to ozone for about 10 minutes for cleaning, and the resulting glass substrate was mounted in a vacuum deposition device;
2) on the ITO anode layer 2, a hole injection layer compound b and a P-type doping compound a were co-evaporated by vacuum evaporation, with a doping ratio of 3% (mass ratio) and a thickness of 5 nm, this resulting layer served as a hole injection layer 3;
3) a compound b was vacuum evaporated on the hole injection layer 3 to a thickness of 100 nm as a first hole transmission layer 4;
4) a hole transmission compound c was vacuum evaporated on the first hole transmission layer 4 to a thickness of 5 nm as a second hole transmission layer 5;
5) a light-emitting host compound d and a doping material compound e were co-vacuum evaporated on the second hole transmission layer 5, with a doping ratio of 3% (mass ratio) and a thickness of 30 nm, as a light-emitting layer 6;
6) a hole blocking layer compound f was vacuum evaporated on the light-emitting layer 6 to a thickness of 5 nm as a hole blocking layer 7;
7) the compound P01 and a N-type doping compound g were vacuum co-evaporated on the hole blocking layer 7, with a doping mass ratio of 1:1 and a thickness of 30 nm, as an electron transmission layer 8;
8) a magnesium-silver electrode was vacuum evaporated on the electron transmission layer 8, with a Mg—Ag mass ratio of 1:9 and a thickness of 10 nm, as the cathode 9; and
9) the compound h was vacuum evaporated on the cathode 9 to a thickness of 65 nm as a covering layer 10.

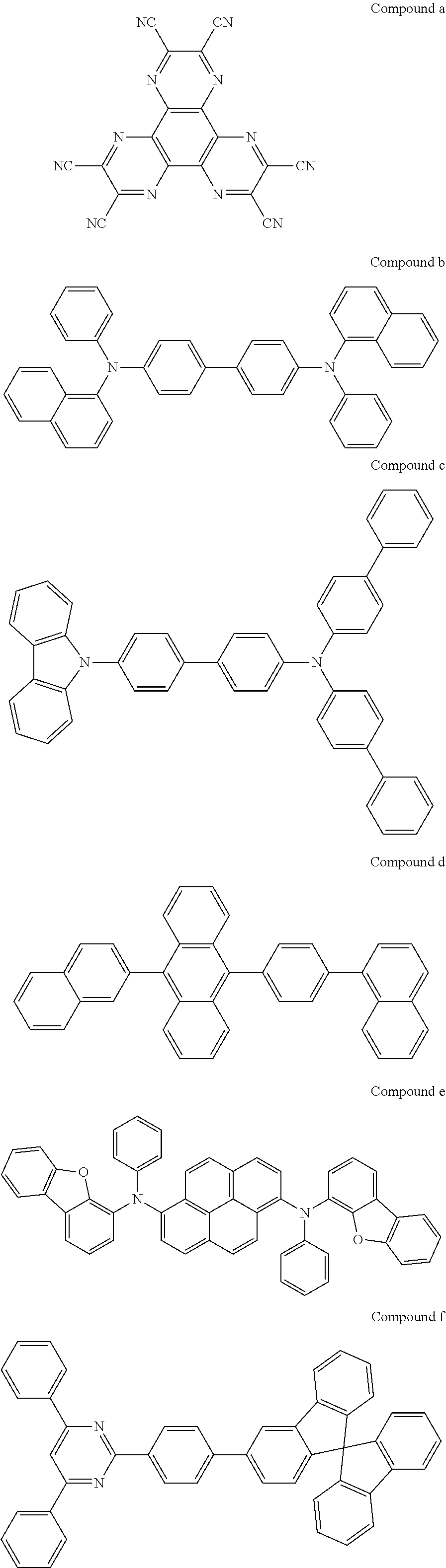

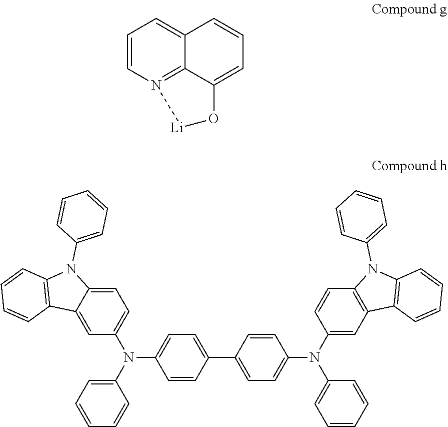

### Application Example 2

This application example was performed as Application Example 1 except that Compound P01 was replaced with Compound P06.

### Application Example 3

This application example was performed as Application Example 1 except that Compound P01 was replaced with Compound P17.

### Application Example 4

This application example was performed as Application Example 1 except that Compound P01 was replaced with Compound P21.

### Application Example 5

This application example was performed as Application Example 1 except that Compound PO1 was replaced with Compound P22.

### Application Example 6

This application example was performed as Application Example 1 except that Compound PO1 was replaced with Compound P23.

### Application Example 7

This application example was performed as Application Example 1 except that Compound P01 was replaced with Compound P26.

### Application Example 8

This application example was performed as Application Example 1 except that Compound P01 was replaced with Compound P33.

### Application Example 9

This application example was performed as Application Example 1 except that Compound PO01 was replaced with Compound P38.

### Application Example 10

This application example was performed as Application Example 1 except that Compound P01 was replaced with Compound P42.

### Application Example 11

This application example was performed as Application Example 1 except that Compound P01 was replaced with Compound P46.

### Application Example 12

This application example was performed as Application Example 1 except that Compound P01 was replaced with Compound P50.

### Comparative Example 1

This Comparative Example 1 adopted the same method as Application Example 1, only except that Compound PO1 was replaced with Comparative Compound 1 as follows:

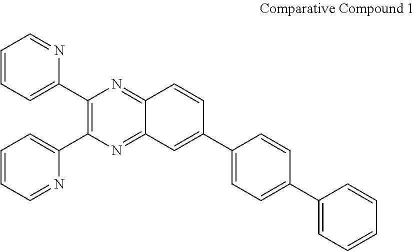 [SMILES: c1ccc(-c2ccc(-c3ccc4nc(-c5ccccn5)c(-c5ccccn5)nc4c3)cc2)cc1]

### Comparative Example 2

This Comparative Example 2 adopted the same method as Application Example 1, only except that Compound PO1 was replaced with Comparative Compound 1 as follows:

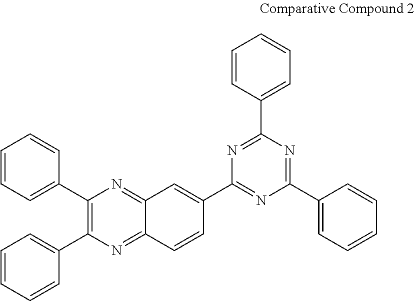 [SMILES: c1ccc(-c2nc(-c3ccccc3)nc(-c3ccc4nc(-c5ccccc5)c(-c5ccccc5)nc4c3)n2)cc1]

### Performance Evaluation of Organic Electroluminescent Device

According to current density and brightness of the organic electroluminescent device at different voltages, operating voltage V and current efficiency CE (cd/A) at a certain current density (10 mA/cm²) were obtained. Service life LT95 (under 20 mA/cm² test condition) was obtained by measuring the duration until brightness of the device decayed to 95% of initial brightness. The test data are shown in Table 6.

TABLE 6

| | | Operat- | | Service |
|---|---|---|---|---|
| | Electron | ing | Current | Life |
| | Transmission | Voltage | Efficiency | LT95 |
| Name of the Device | Layer | (V) | (cd/A) | (h) |
| Application Example 1 | Compound P01 | 3.42 | 4.86 | 118 |
| Application Example 2 | Compound P06 | 3.38 | 5.65 | 152 |
| Application Example 3 | Compound P17 | 3.41 | 5.76 | 148 |
| Application Example 4 | Compound P21 | 3.48 | 5.34 | 136 |
| Application Example 5 | Compound P22 | 3.56 | 5.23 | 138 |
| Application Example 6 | Compound P23 | 3.51 | 5.28 | 135 |
| Application Example 7 | Compound P26 | 3.42 | 5.51 | 142 |
| Application Example 8 | Compound P33 | 3.45 | 5.54 | 145 |
| Application Example 9 | Compound P38 | 3.44 | 5.42 | 134 |
| Application Example 10 | Compound P42 | 3.39 | 5.23 | 146 |
| Application Example 11 | Compound P46 | 3.54 | 5.35 | 133 |
| Application Example 12 | Compound P50 | 3.57 | 5.25 | 139 |
| Comparative Example 1 | Comparative | 3.98 | 3.54 | 92 |
| | Compound 1 | | | |
| Comparative Example 2 | Comparative | 4.02 | 3.45 | 87 |
| | Compound 2 | | | |

It can be seen from the results in Table 6 that the organic compound of the present disclosure can be applied as an electron transmission layer material of an organic electroluminescent device, and has a lower operating voltage, a higher luminous efficiency and a longer service life. This is at least in part because the compound of the present disclosure has a deeper LUMO energy level, and has a smaller difference in band gap with the LUMO energy level of the adjacent material, which is more conducive to the injection of electrons. Meanwhile, higher electron mobility is conducive to transmission of electrons, balancing electrons and holes and bringing higher luminous efficiency.

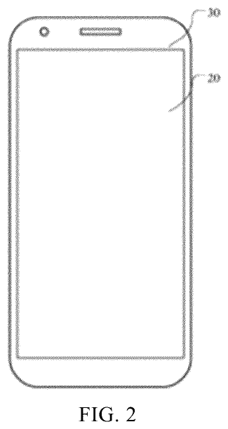

The present disclosure further provides a display device, which includes the organic light-emitting display panel as described above. In the present disclosure, the organic light-emitting device can be an OLED, which can be used in an organic light-emitting display device, where the organic light-emitting display device may be a mobile phone display screen, a computer display screen, a television display screen, a smart watch display screen, a smart automobile display panel, a VR or AR helmet display screen, display screens of various smart devices, etc. FIG. 2 is a schematic diagram of a display device according to an embodiment of the present disclosure. In FIG. 2, 20 represents a mobile phone display panel, and 30 represents a display device.

The applicant states that the present disclosure describes the method and the core idea of the present disclosure through the above embodiments, but the present disclosure is not limited to the above embodiments, that is, it does not mean that the present disclosure must depend on the above embodiments for implementation. It will be apparent to those skilled in the art that any improvements made to the present disclosure, equivalent replacements to the raw materials of the products of the present disclosure and addition of adjuvant ingredients, and choices of the specific implementations, etc., all fall within the protection scope and the disclosure scope of the present disclosure.

What is claimed is:

1. A quinoxaline compound, having a chemical structure represented by Formula I: 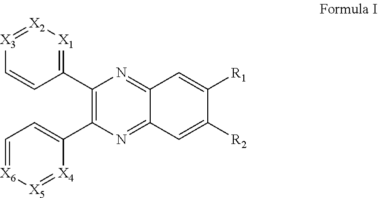
wherein R₁ and R₂ are each independently selected from hydrogen, substituted or unsubstituted C3-C60 aryl, substituted or unsubstituted C3-C60 heteroaryl; X₁, X₂, X₃, X₄, X₅, and X₆ are each independently selected from methylene or nitrogen atom, and at least one of X₁, X₂, X₃, X₄, X₅, and X₆ comprises an N atom.

2. The quinoxaline compound according to claim 1, wherein R₁ and R₂ are each independently selected from C3-C60 aza-aromatic group.

3. The quinoxaline compound according to claim 1, wherein R₁ and R₂ are the same or different.

4. The quinoxaline compound according to claim 1, wherein X₁, X₂, X₃, X₄, X₅, and X₆ are each independently selected from methylene or nitrogen atoms, and one of X₁, X₂, X₃, X₄, X₅, and X₆ comprises an N atom.

5. The quinoxaline compound according to claim 1, wherein R₁ and R₂ are each independently selected from any one of the following groups: 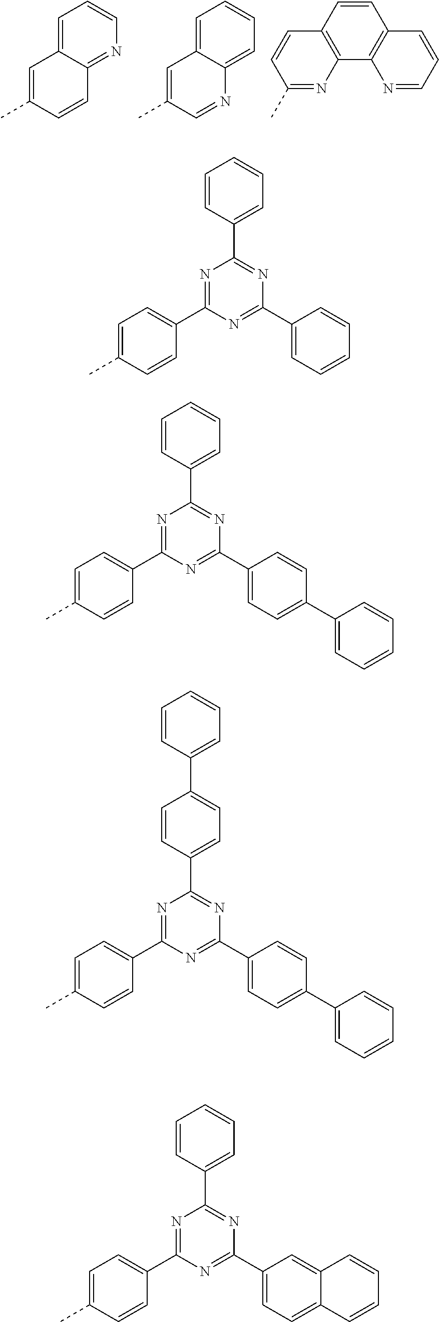 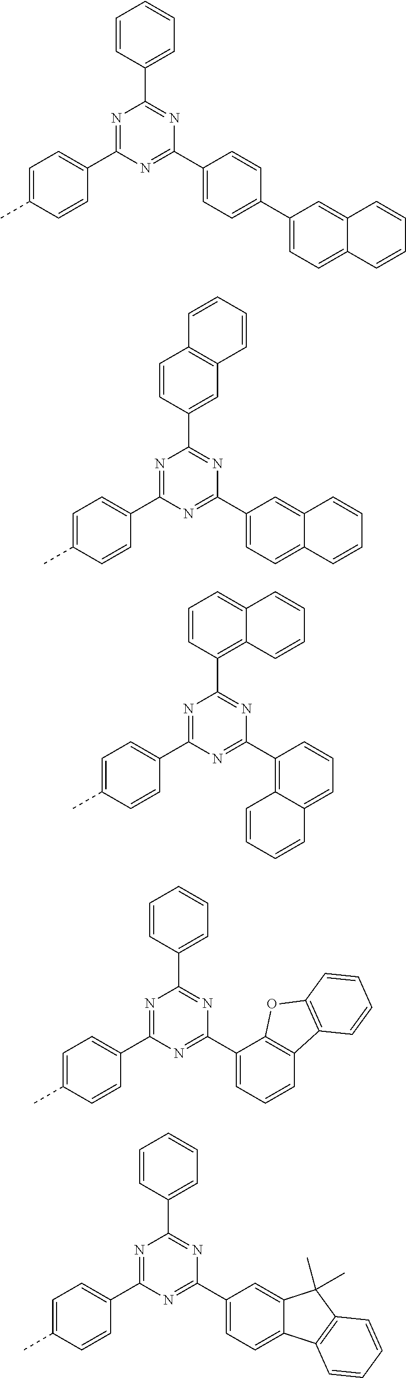 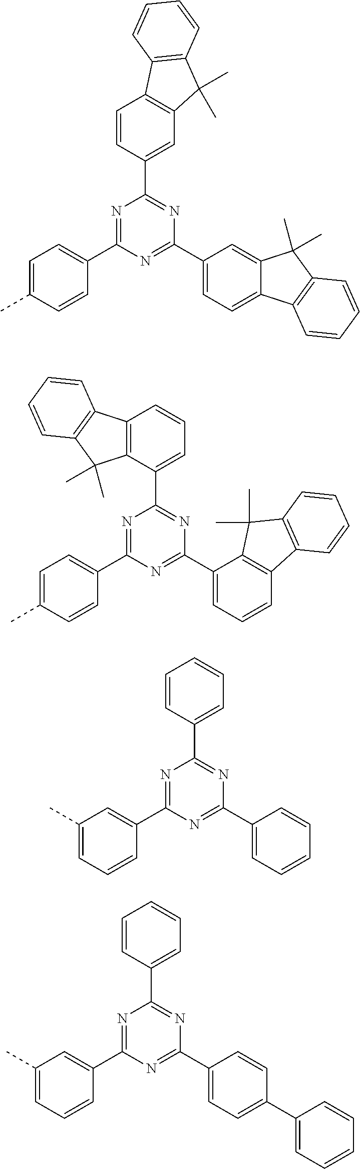 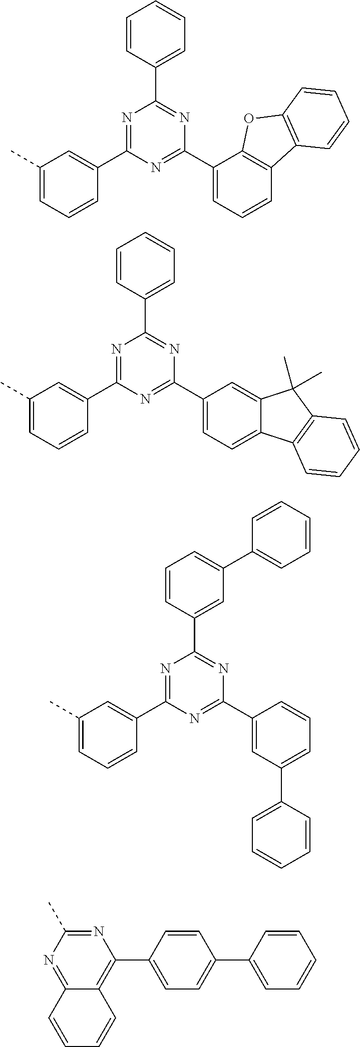 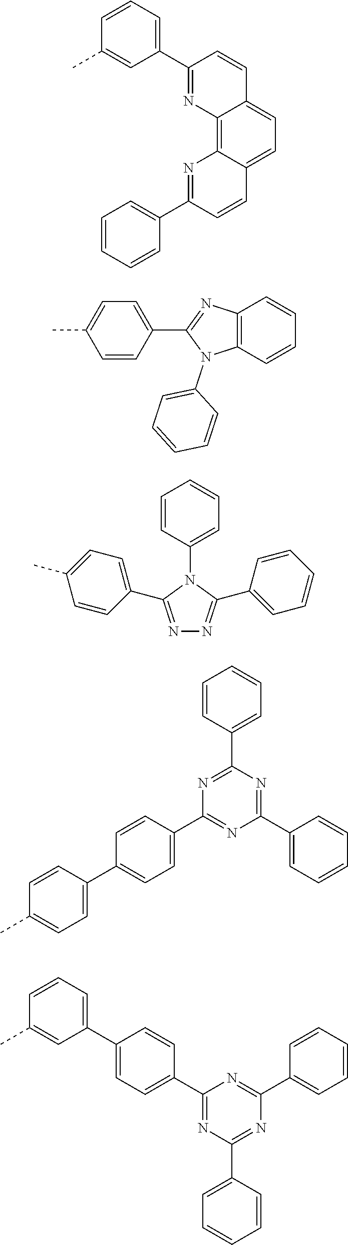 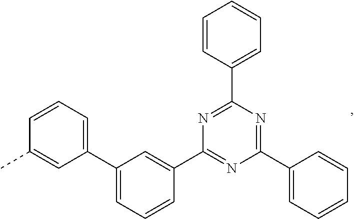
wherein the dashed line represents a bonding site of the group.

6. The quinoxaline compound according to claim 1, wherein the compound is selected from the following chemical structures:

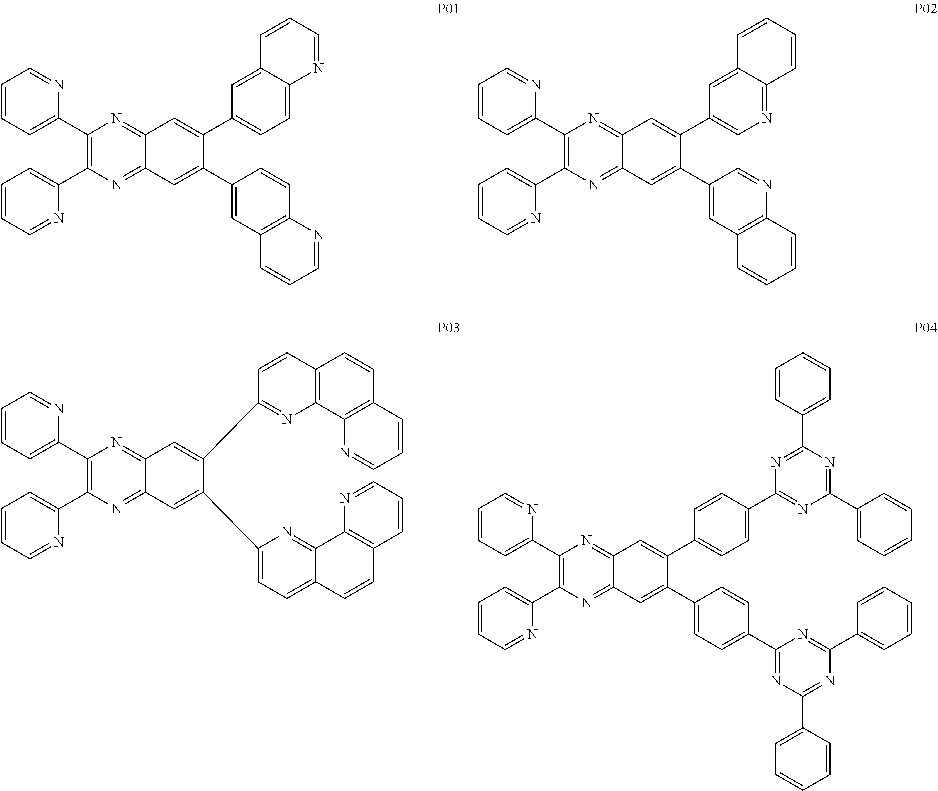 [SMILES: c1ccc(-c2nc3cc(-c4ccc5ncccc5c4)c(-c4ccc5ncccc5c4)cc3nc2-c2ccccn2)nc1 ; c1ccc(-c2nc3cc(-c4cnc5ccccc5c4)c(-c4cnc5ccccc5c4)cc3nc2-c2ccccn2)nc1 ; c1ccc(-c2nc3cc(-c4ccc5ccc6cccnc6c5n4)c(-c4ccc5ccc6cccnc6c5n4)cc3nc2-c2ccccn2)nc1 ; c1ccc(-c2nc(-c3ccccc3)nc(-c3ccc(-c4cc5nc(-c6ccccn6)c(-c6ccccn6)nc5cc4-c4ccc(-c5nc(-c6ccccc6)nc(-c6ccccc6)n5)cc4)cc3)n2)cc1]

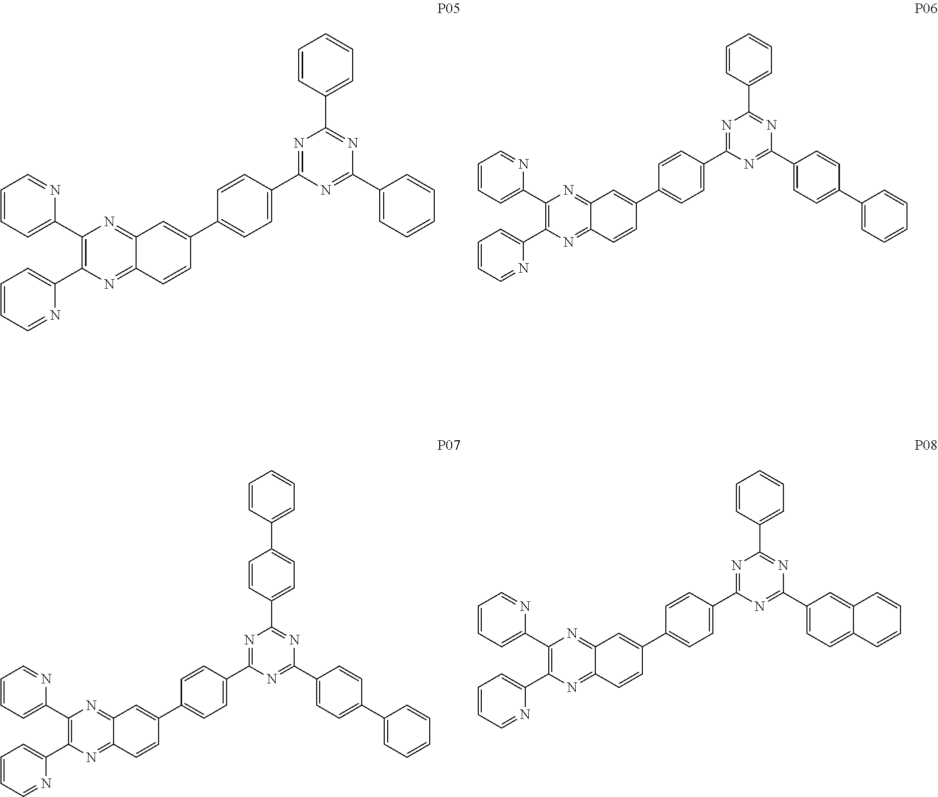 [SMILES: c1ccc(-c2nc(-c3ccccc3)nc(-c3ccc(-c4ccc5nc(-c6ccccn6)c(-c6ccccn6)nc5c4)cc3)n2)cc1 ; c1ccc(-c2ccc(-c3nc(-c4ccccc4)nc(-c4ccc(-c5ccc6nc(-c7ccccn7)c(-c7ccccn7)nc6c5)cc4)n3)cc2)cc1 ; c1ccc(-c2ccc(-c3nc(-c4ccc(-c5ccccc5)cc4)nc(-c4ccc(-c5ccc6nc(-c7ccccn7)c(-c7ccccn7)nc6c5)cc4)n3)cc2)cc1 ; c1ccc(-c2nc(-c3ccc(-c4ccc5nc(-c6ccccn6)c(-c6ccccn6)nc5c4)cc3)nc(-c3ccc4ccccc4c3)n2)cc1]

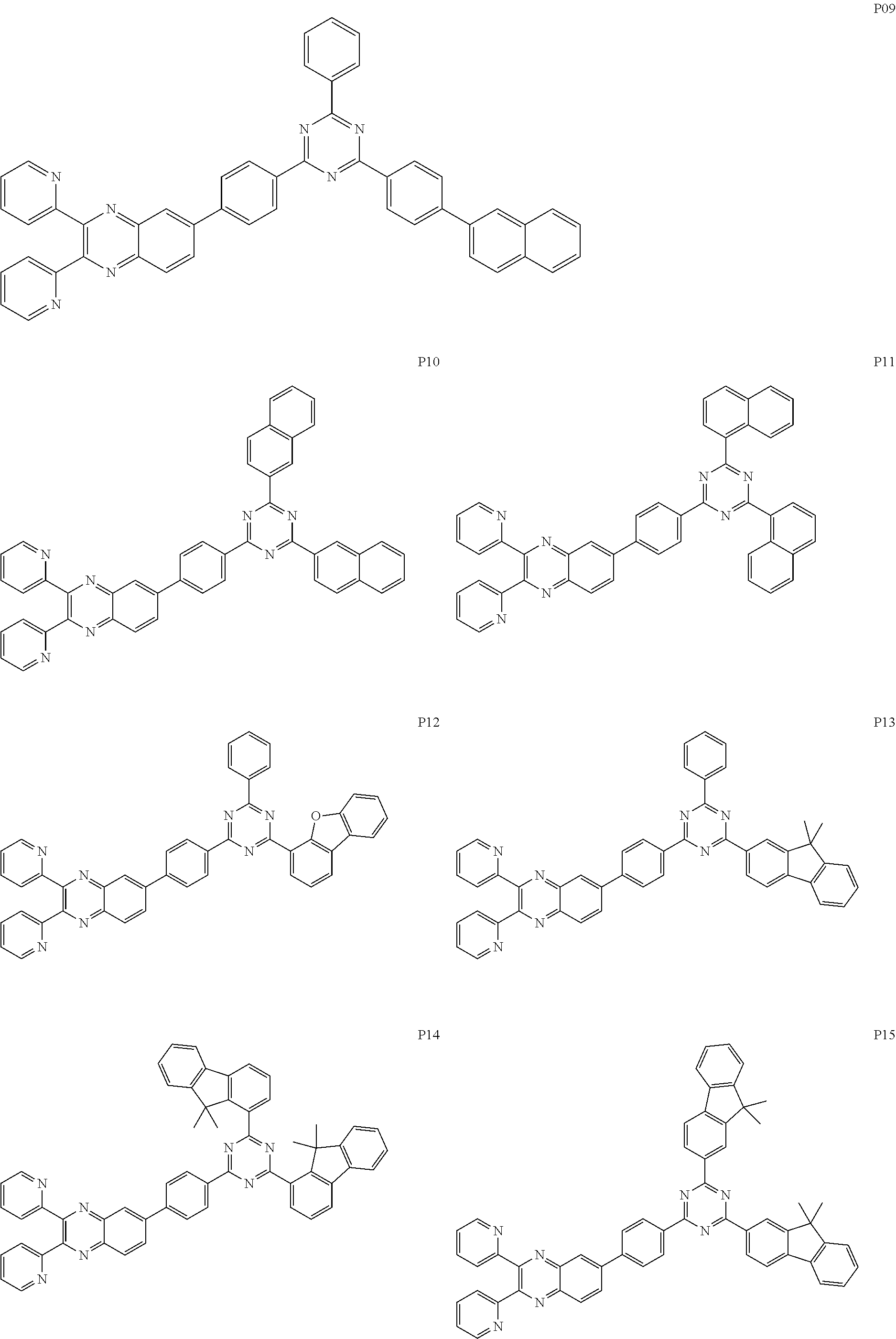

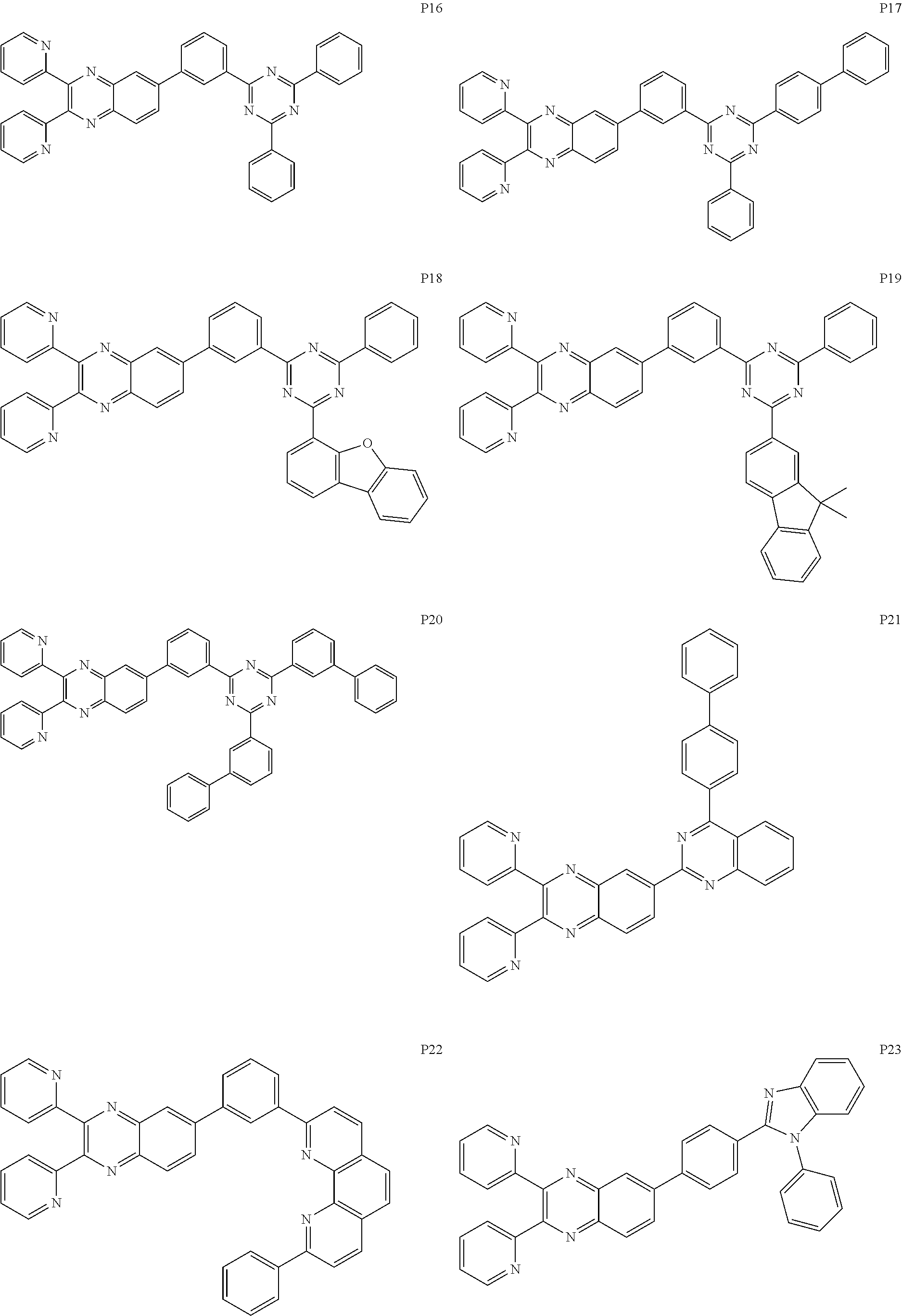

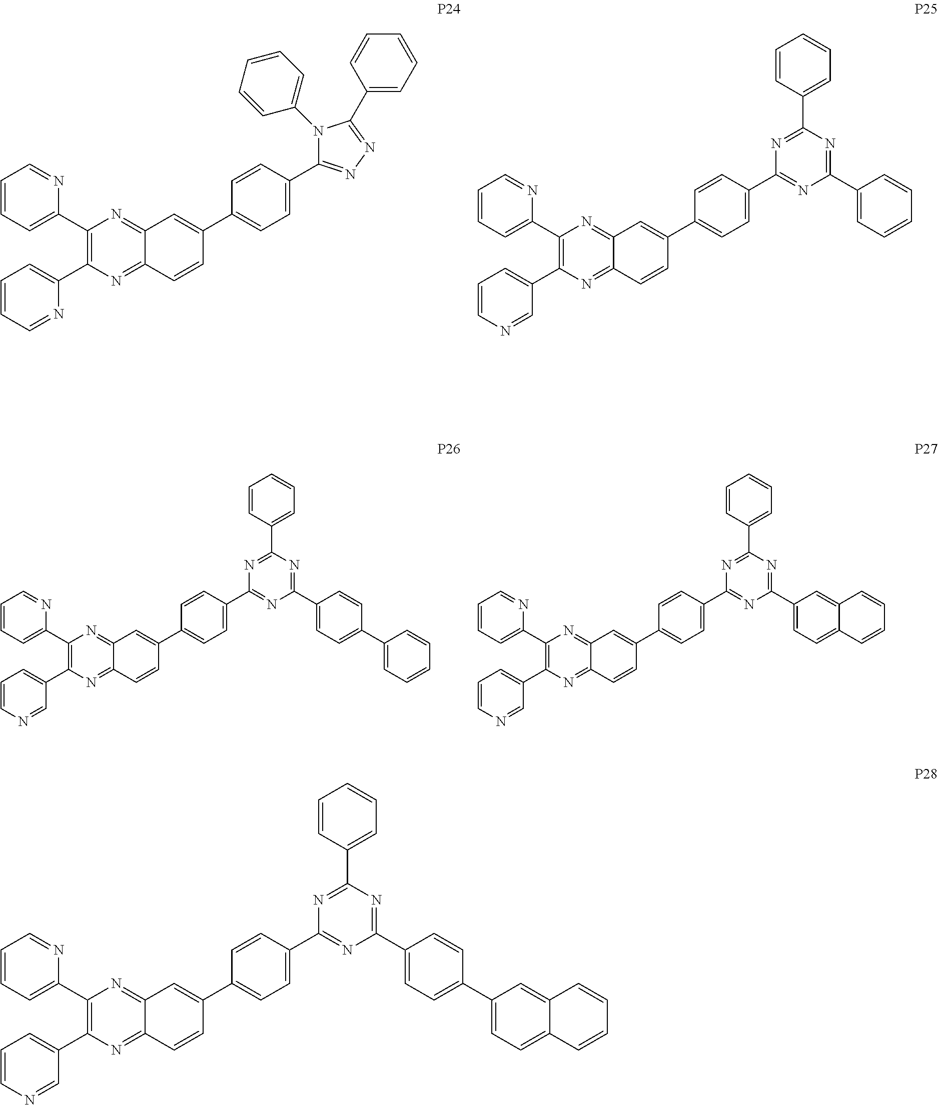 [SMILES: c1ccc(-c2nnc(-c3ccc(-c4ccc5nc(-c6ccccn6)c(-c6ccccn6)nc5c4)cc3)n2-c2ccccc2)cc1 ; c1ccc(-c2nc(-c3ccccc3)nc(-c3ccc(-c4ccc5nc(-c6cccnc6)c(-c6ccccn6)nc5c4)cc3)n2)cc1 ; c1ccc(-c2ccc(-c3nc(-c4ccccc4)nc(-c4ccc(-c5ccc6nc(-c7cccnc7)c(-c7ccccn7)nc6c5)cc4)n3)cc2)cc1 ; c1ccc(-c2nc(-c3ccc(-c4ccc5nc(-c6cccnc6)c(-c6ccccn6)nc5c4)cc3)nc(-c3ccc4ccccc4c3)n2)cc1 ; c1ccc(-c2nc(-c3ccc(-c4ccc5ccccc5c4)cc3)nc(-c3ccc(-c4ccc5nc(-c6cccnc6)c(-c6ccccn6)nc5c4)cc3)n2)cc1]

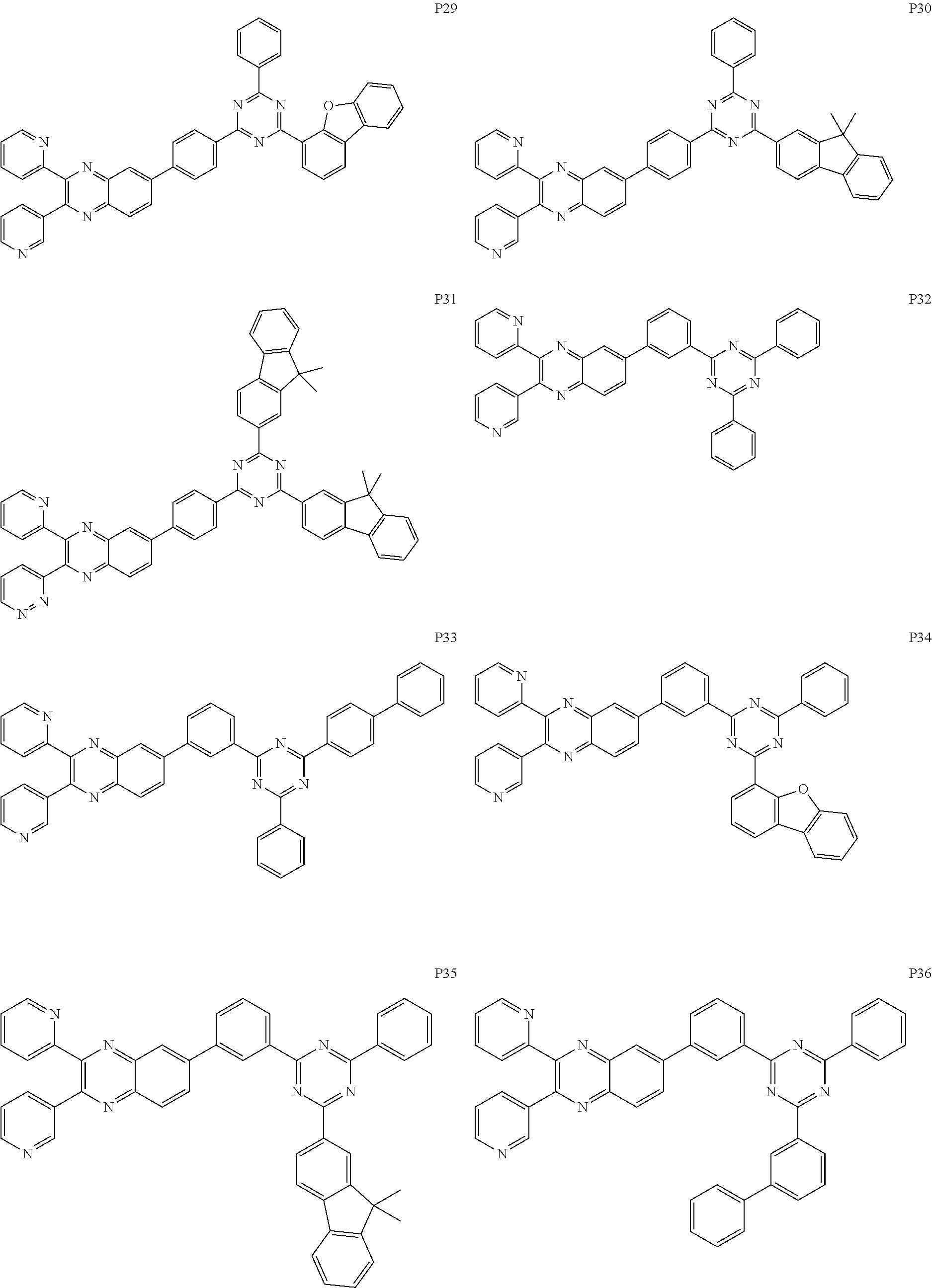

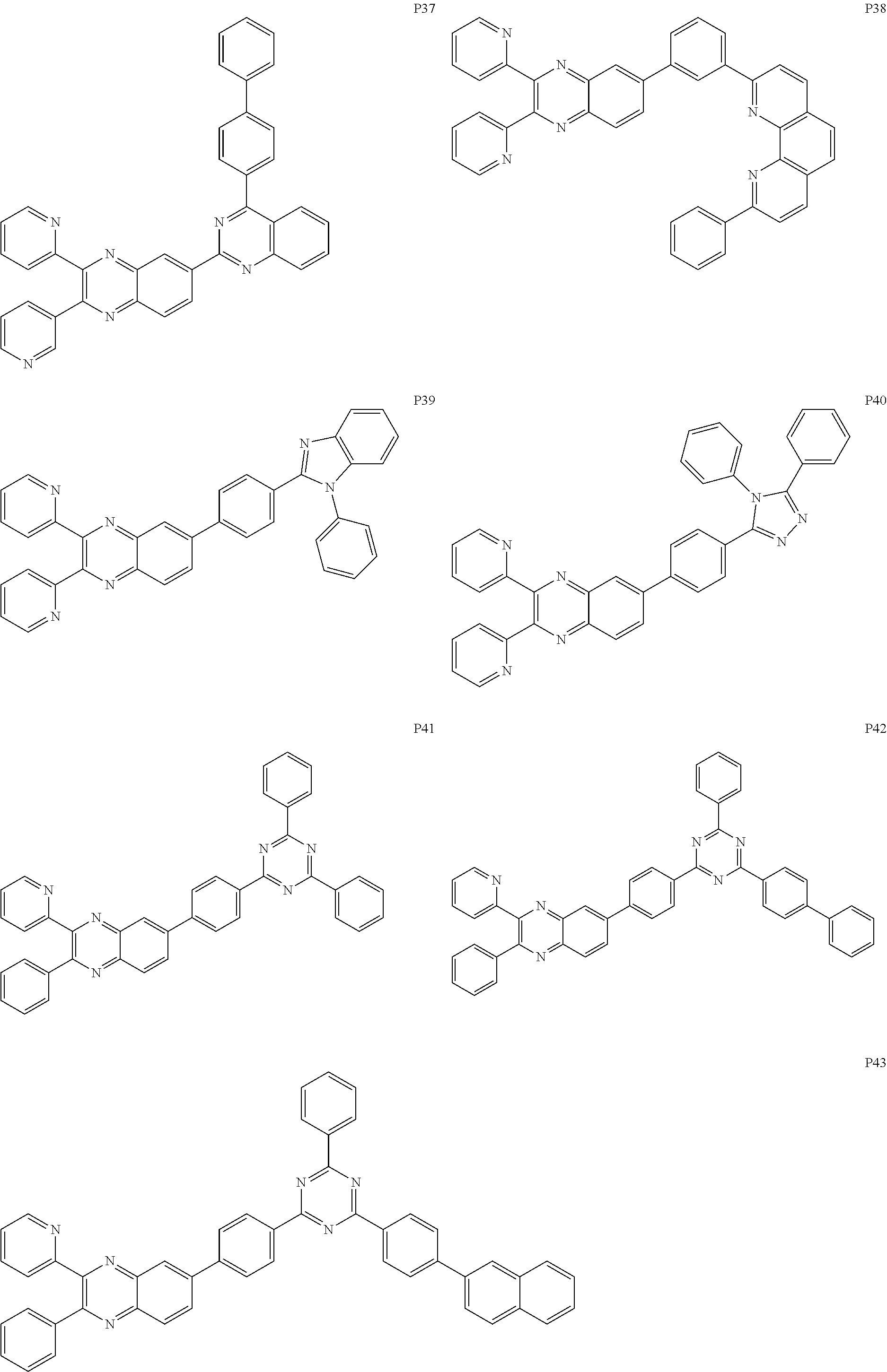

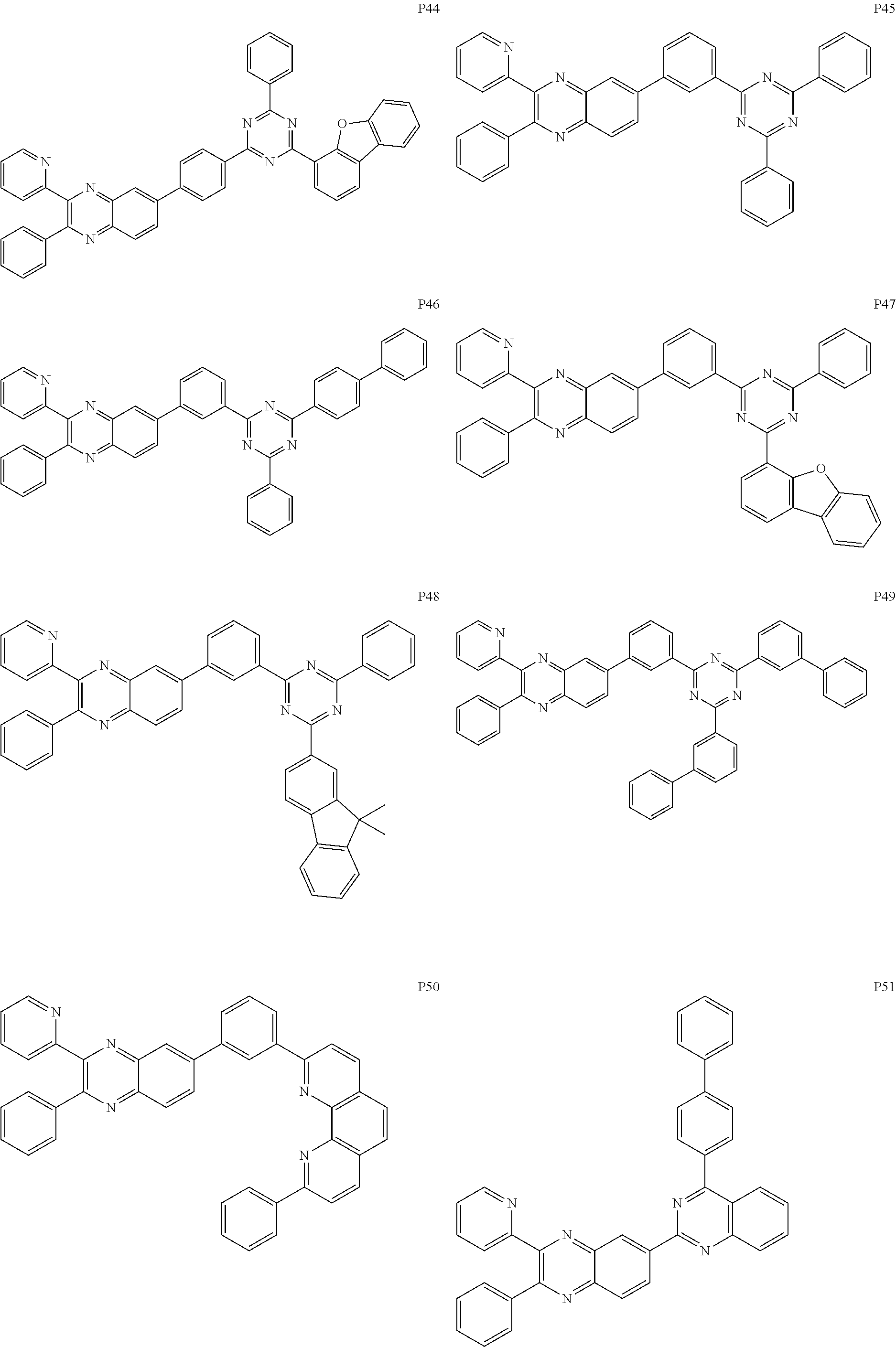

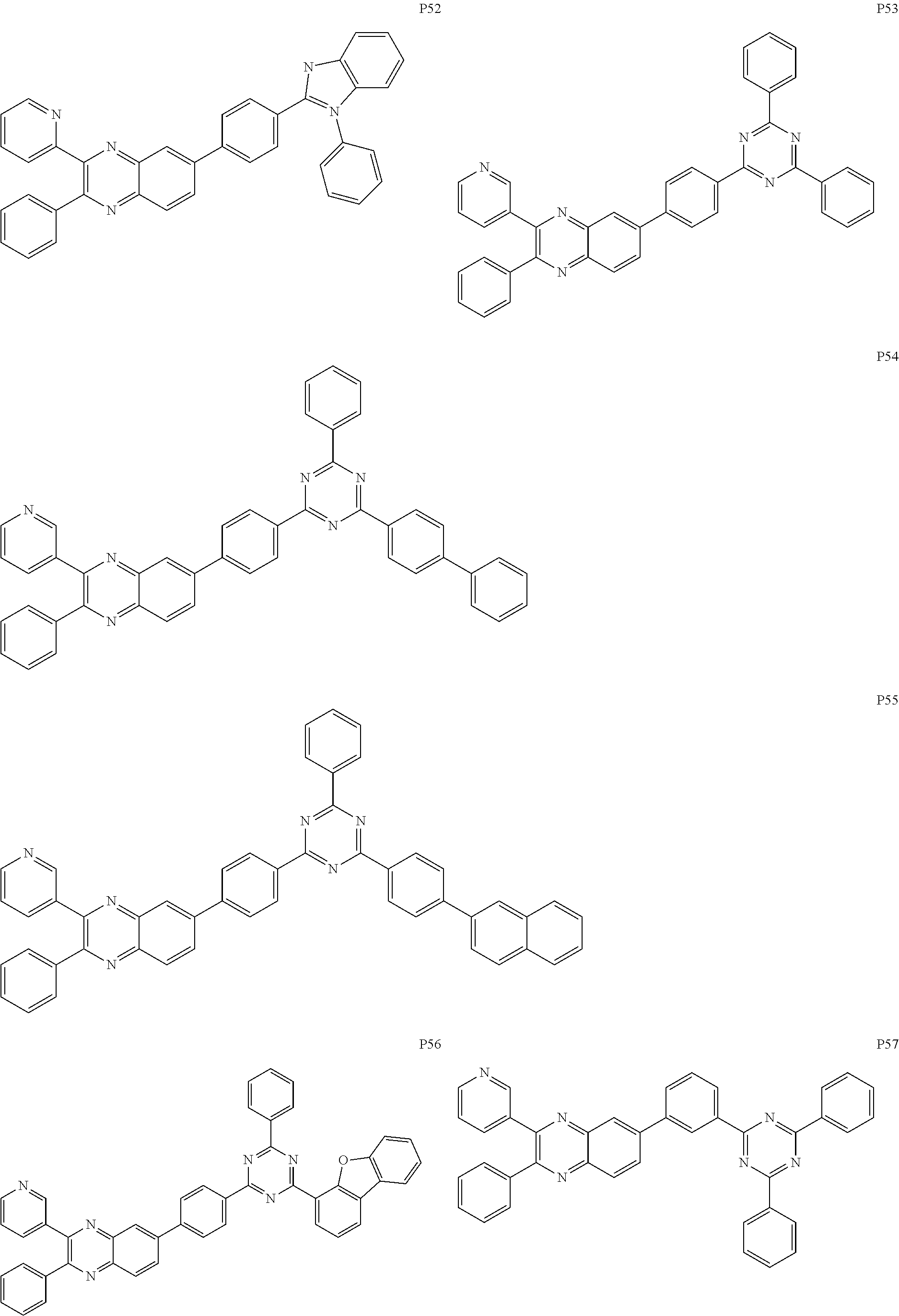

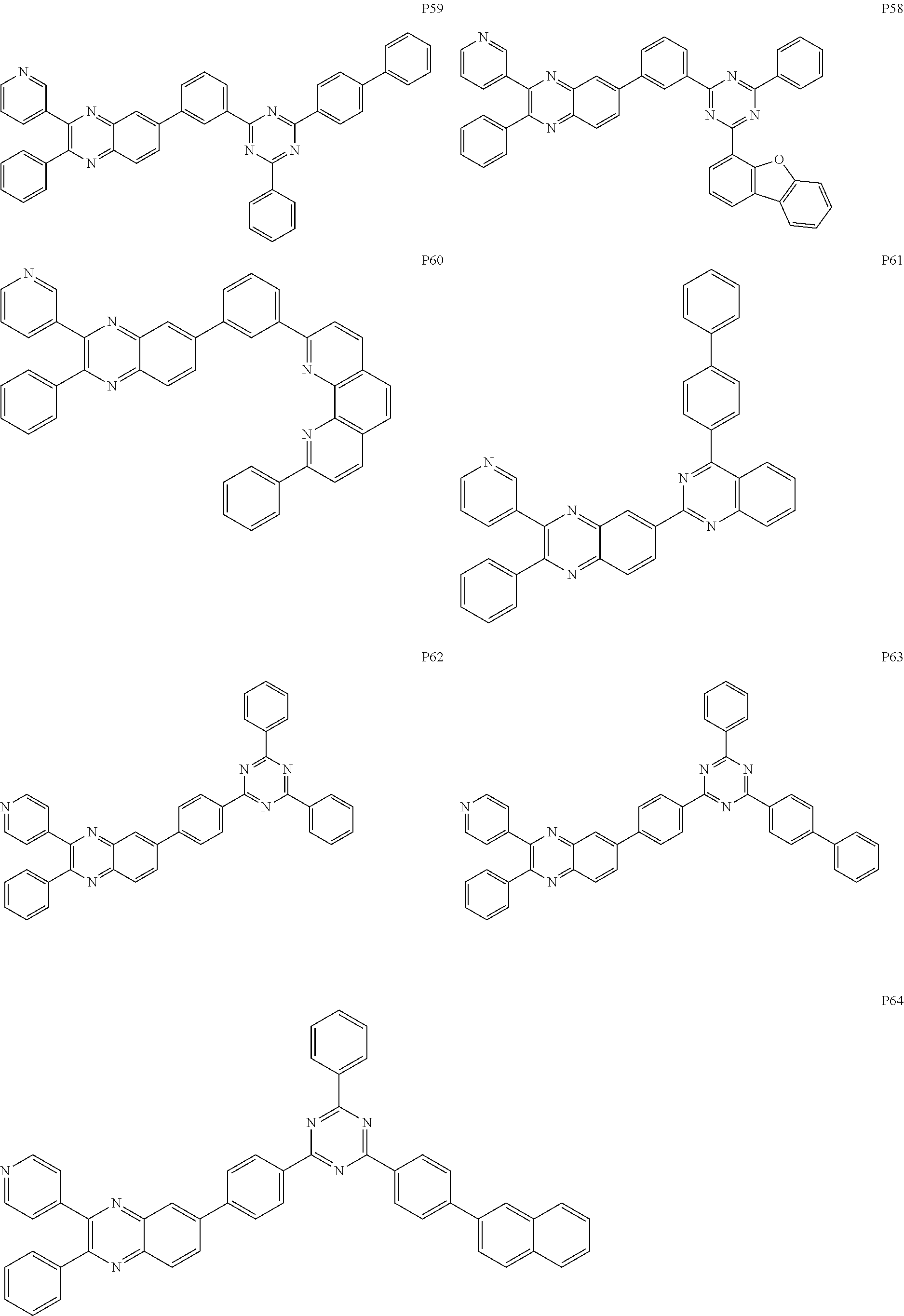

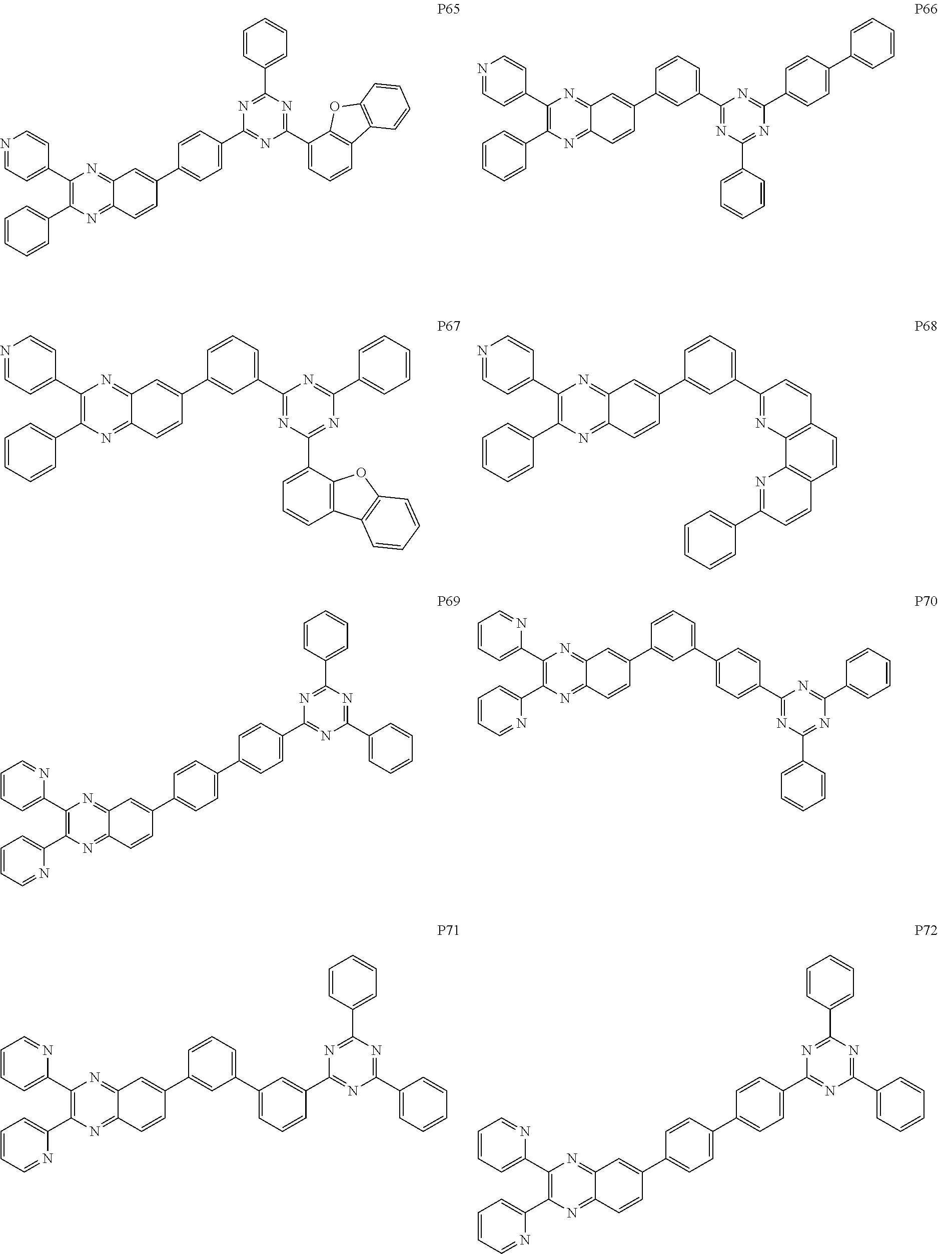

7. An electron transmission material, comprising a quinoxaline compound according to claim 1.

8. An organic electroluminescent device, comprising a cathode, an anode, and an organic thin film layer located between the cathode and the anode, wherein the organic thin film layer comprises an electron transmission layer, and the electron transmission layer comprises at least one quinoxaline compound according to claim 1.
